(19) United States
(12) Patent Application Publication
(10) Pub. No.: US 2024/0017075 A1
(43) Pub. Date: Jan. 18, 2024

(54) SYSTEMS AND METHODS TO COMPENSATE FOR WAVEFORM DISTORTION

(71) Applicant: Advanced Neuromodulation Systems, Inc., Plano, TX (US)

(72) Inventors: Hyun-Joo Park, Frisco, TX (US); Yagna Pathak, Skokie, IL (US)

(21) Appl. No.: 18/221,373

(22) Filed: Jul. 12, 2023

Related U.S. Application Data
(60) Provisional application No. 63/389,761, filed on Jul. 15, 2022.

(51) Int. Cl. A61N 1/36

(52) U.S. Cl. CPC A61N 1/36189; A61N 1/36139; A61N 1/36071

(57) ABSTRACT

Devices and methods to effectuate waveform distortion adjusted stimulation therapy of tissue, based on an inverse model of the tissue, are disclosed. In some aspects a linear model of an electrode used to delivery therapy and of the tissue is determined. Parameter values of the linear model may be determined based on measuring voltage feedback during application of stimulation and/or measurement waveforms. The parameter values of the linear model of the electrode and tissue may be used to determine a forward model of the electrical response of the tissue. From the forward model, an inverse model of the tissue may be determined and the inverse model can be used to modify or filter a stimulation waveform to reduce waveform distortion caused by tissue, such as tissue primitive and/or tissue capacitance. Accordingly, providing therapy with the filtered waveform may have increased efficacy by accounting for and adjusting for waveform distortion.

### CROSS-REFERENCE TO RELATED APPLICATIONS

The present application claims the benefit of priority from U.S. Provisional Application 63/389,761 filed on Jul. 15, 2022, the contents of which are incorporated herein by reference in their entirety.

### TECHNICAL FIELD

The present invention relates generally to stimulation systems and more specifically, to open and closed loop therapy stimulation systems utilizing a stimulation waveform modified based on an inverse model of the tissue to apply therapy to a patient.

### BACKGROUND OF THE INVENTION

Stimulation therapy devices provide electrical stimulation therapy to treat a wide variety of medical conditions. Such devices can be used internally or externally. For example, implantable medical devices (IMDs) may be implanted within a patient's body and provide functionality to treat medical conditions internally. To illustrate, IMDs may be used to control delivery of electrical stimulation pulses or signals to a targeted tissue (e.g., brain tissue, muscle tissue, nerves, etc.) of a patient to treat pain, movement disorders (e.g., Parkinson's disease), epilepsy and seizures, or other conditions of the patient (e.g., cardiac pace making, cardiac rhythm management, treatments for congestive heart failure, implanted defibrillators, incontinence, depression, and the like). The IMDs generally include an implantable pulse generator (IPG) that generates electrical pulses or signals that are transmitted to a targeted tissue or nerves through a therapy delivery element, such as a lead having one or more electrodes. The therapy delivery element is generally placed within the patient's body to achieve therapeutic efficacy or reduced side effects. For example, therapy delivery elements in the form of leads are commonly implanted along peripheral nerves, within the epidural or intrathecal space of the spinal column, and around the heart, brain, or other organs or tissue of a patient. Once implanted, the lead extends from the stimulation site to the location of the implantable electrical stimulation device.

Traditionally, the aforementioned electrodes are principally deployed for providing electrical stimulation to one or more parts of an anatomy of a patient. For example, in pain management applications electrical stimulation may be provided via electrodes disposed proximate to tissue of the patient's spinal cord (e.g., spinal cord stimulation (SCS)), such as a dorsal root ganglion (DRG). A DRG is a cluster of neurons in a dorsal root of a spinal nerve.

While stimulation therapy devices provide various ways to treat a multitude of tissue types, distortion of stimulation therapy waveforms (hereinafter “waveform distortion”) has proved challenging due, in part, to tissue permittivity and/or tissue capacitance. Waveform distortion can make it challenging to actually measure the stimulation effect or volume of tissue affected by properties of the tissue itself interfering with the collection of adequate sensory data after delivery of the electrical stimulation pulses. Lack of reliable feedback data has impeded the compensation of waveform distortion and the use of closed loop control of stimulation devices for certain types of therapies. An additional challenge is that electrodes are usually positioned to optimize lead manufacturability and not necessarily to optimize delivery of electrical stimulation pulses to particular anatomical regions of a patient or to provide stimulation pulse capabilities specific to a target region of the patient's anatomy. As such, existing technologies for recording sensory data may be insufficient to record sensory data suitable for use in waveform distortion correction systems (e.g., due to noise, artifacts, etc.).

### BRIEF SUMMARY OF THE INVENTION

Biological tissue can have a high permittivity, and permittivity of the biological tissue depends on a frequency of the signal applied to (e.g., passing through) the tissue. This frequency dependent high permittivity may cause distortion of voltage experienced by and/or measured from the tissue when certain waveforms of electrical stimulation are used, such as constant current waveforms. When electrical stimulation is applied, neurons in the biological tissue are activated in proportion to a second derivative of the induced extracellular potentials by electrical stimulation. The distortion of the stimulation waveform experienced by the neurons can potentially cause errors in estimation of a volume of tissue activation caused by the stimulation waveform, such as when square pulses are used. When non-rectangular pulse waveforms are used for stimulation, such as a noise waveform, the tissue capacitance can potentially cause significant shift of the frequency components of the stimulation waveform and alter the therapy. This altered therapy may reduce the efficacy of the treatment and/or treat a larger or smaller area than intended.

In the aspects described herein, devices and methods are described to reduce waveform distortion experienced during electrical stimulation. The aspects described herein may enable compensation for tissue permittivity and/or capacitance so that the stimulation waveform experienced by the target tissue more closely resembles the desired stimulation waveform and has reduced distortion. In some aspects, a waveform may be modified or filtered, such as prefiltered, before application of the stimulation therapy based on tissue capacitance and/or tissue permittivity of the target tissue. Prefiltering of the stimulation waveform may enable the therapy device to be less complex. For example, a separate measurement waveform generator and sensing/feedback circuitry may not be needed when prefiltering is used.

As an illustrative example, a voltage profile experienced by the tissue may be distorted because of the capacitive component of tissue even with current controlled stimulation. Because the activating function of the tissue is proportional to the second spatial derivative of the extracellular potentials, stimulation waveform distortion can cause significant difference between the stimulation waveform provided by the device and the stimulation waveform actually experienced by the target tissue. Filtering the stimulation waveform using an inverse model of the tissue can make the stimulation waveform that the tissue experiences closer to the original target stimulation waveform, and thus much more effective. In the aspects described herein, the inverse model may be obtained by estimation of the parameters of the forward model and finding the inverse transfer function of the forward model. For simplicity, a linear model composed of resistors and capacitors may be used to estimate the tissue and electrode model, and using curve fitting methods with experimental data, the model parameters (e.g., resistance of the resistors and capacitance of the capacitors) of the linear model are obtained.

In an exemplary aspect, a linear model is generated to approximate the electrical characteristics of an electrode of the therapy device and of the tissue to be treated, also referred to herein as target tissue. As illustrative, non-limiting examples, the linear model may approximate or model the electrode as a capacitor and may approximate or model the tissue as a resistor and capacitor in parallel, with the electrode coupled in series with the tissue. In another illustrative example, the electrode can be approximated or modeled as capacitor and resistor in parallel, which are coupled in series with the model of the tissue (e.g., a resistor and capacitor in parallel). The capacitor in the electrode model may be used to represent double layer capacitance and the resistor may be used to represent Faradaic reaction. At low stimulation amplitudes, the Faradaic reaction may be low or negligible, and thus a capacitor model for electrode may be appropriate for some tissues and/or therapies.

After the linear model is obtained, the values, also referred to as parameter values), of the elements (e.g., values of the capacitors and resistors) of the linear model are obtained by measurement. For example, voltage measurements of the target tissue may be obtained during application of a measurement waveform. In a first example, electrochemical impedance spectroscopy (EIS) may be used to determine the parameter values. When using EIS, a low amplitude sinusoidal signal current is injected into the target tissue, and the voltage of the target tissue is measured. A magnitude and phase at each frequency of the low amplitude sinusoidal signal is calculated by dividing the measured voltage by the input current of the low amplitude sinusoidal signal. Then the values of the resistors and capacitors are estimated to minimize the error between the model and the measured EIS data, such as by using best fit and/or regression techniques. In a second example, rectangular pulses of different pulse widths may be used. To illustrate, a first pulse with a first pulse width (or duty cycle) may be applied and then a second pulse with a second pulse width may be applied which is narrower (e.g., shorter) or wider (e.g., longer) than the first pulse width. One or more measurements may be obtained for or during each pulse. A profile of the voltage for a current stimulus pulse is measured, and the measured data is used to estimate the model parameters similar to the EIS method.

After the component values of the linear model as a whole are obtained, a forward model of the tissue itself may be obtained. The forward model may represent the voltage across the tissue over a given current injection. When correcting for the voltage across the tissue for a given current controlled stimulation, the forward model of the voltage of the tissue may be used to correct for any waveform distortion, and particularly voltage distortion. As the model of the tissue and electrode is linear (e.g., has a linear response), the forward model of the tissue can be easily obtained in the frequency domain by dividing the voltage across the tissue by the total transfer function.

After the forward model of the tissue itself is obtained, an inverse model of the tissue itself may be obtained. The inverse model G(s) may be a mathematical inverse of the forward model F(s) in the frequency domain. The inverse model of the tissue represents what the current stimulation waveform profile should be for a given desired voltage waveform. Said another way, the inverse model may be used to modify the stimulation waveform actually applied to the tissue such that stimulation waveform experienced by the tissue better matches the desired stimulation waveform.

The stimulation waveform may then be modified or filtered by the inverse model of the tissue before application of therapy to the target tissue to reduce distortion of the waveform that is experienced by the tissue. Additionally, or alternatively, the stimulation waveform may be modified or filtered by the inverse model (or an updated inverse model) during application of the stimulation therapy. This modification or filtering may include amplifying high frequency signals or components thereof. Additionally or alternatively, modification or filtering may include attenuating lower frequency signals or components thereof to a greater extent than high frequency signals or components thereof.

Use of the inverse model may compensate for the distortion caused by the capacitive property of the tissue. Thus, the voltage experienced by the target tissue may be closer to the original stimulation waveform as compared to unmodified stimulation waveforms. Typically, the tissue permeability is high at low frequency, and the inverse filter will have higher amplitude at high frequency compared to low frequency.

The aspects described herein may be used with open loop therapy systems and/or less complex therapy systems. For example, the stimulation therapy may be prefiltered based on another device and a lower complexity device or implanted device may be able to apply undistorted or less distorted therapy. Additionally, the aspects described herein may be used with closed loop therapy systems and/or complex therapy systems. For example, in some implementations, the measurement waveform is a first stimulation waveform and a feedback loop may be used to adjust the stimulation waveform to a second stimulation waveform based on an inverse model of the tissue. As another example, the a conventional closed loop therapy system may be used with an inverse model prefiltering system. Accordingly, the aspects described herein can be applied to all therapy systems to reduce waveform distortion when applying therapy to tissue.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 illustrates a block diagram illustrating aspects of a therapy system according to embodiments of the present disclosure according to embodiments of the present invention;

FIG. 2 illustrates a block diagram illustrating aspects of another therapy system according to embodiments of the present disclosure according to embodiments of the present invention;

FIG. 3 is an example of a linear model to approximate an electrode of a therapy device and tissue;

FIG. 4 is another example of a linear model to approximate an electrode of a therapy device and tissue;

FIG. 5 depicts exemplary amplitude and phase graphs of voltage measurements and of model estimations for linear models of an electrode of a therapy device and tissue;

FIG. 6 depicts exemplary forward and inverse voltage models for tissue for a particular stimulation waveform;

FIG. 7 is a flowchart depicting an exemplary process for providing therapy which accounts for waveform distortion according to embodiments of the present disclosure;

FIG. 8 is a flowchart depicting another exemplary process for providing therapy which accounts for waveform distortion according to embodiments of the present disclosure;

FIG. 9 is a flowchart depicting an exemplary process for determining an inverse model of tissue according to embodiments of the present disclosure;

FIG. 10 is a flowchart depicting an exemplary process for determining parameter values of a linear model according to embodiments of the present disclosure;

FIG. 11 is a flowchart depicting an exemplary process for providing closed loop electrical stimulation based on an inverse model of tissue to according to embodiments of the present disclosure; and

FIG. 12 is a block diagram of an example of a device that supports filtering stimulation waveforms based on an inverse model of tissue according to one or more aspects of the of the present disclosure.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular embodiments illustrated herein.

### DETAILED DESCRIPTION OF THE INVENTION

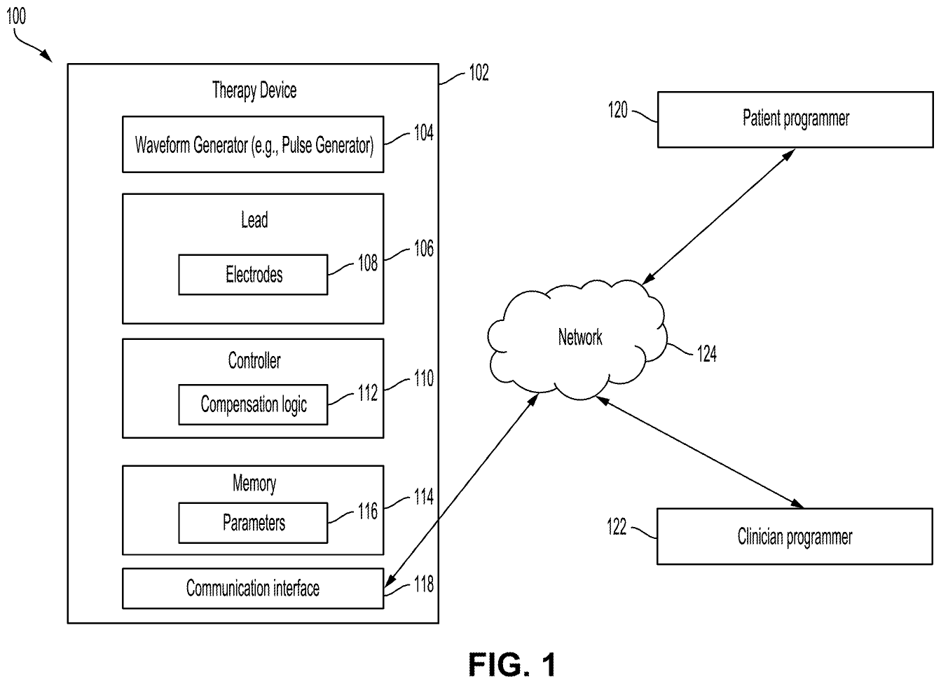

Referring to FIG. 1, a block diagram illustrating aspects of a therapy system according to embodiments of the present disclosure is shown as system 100. System 100 (e.g., a neurostimulation system) may be deployed to provide electrical stimulation, such as may be delivered to nervous system tissue of a patient to treat a condition or to spinal tissue of the patient to mitigate pain. The nervous system tissue may include brain tissue, neurons, axions, ganglions, nerves, etc. Additionally or alternatively, the therapy device may deliver therapy to other tissues, such as organ tissue, muscle tissue, etc. System 100 may be configured to compensate for waveform distortion caused by tissue. For example, system 100 may provide stimulation therapy which is distortion resistant or distortion adjusted to account for or mitigate against tissue permittivity and/or tissue capacitance.

As shown in FIG. 1, system 100 includes a therapy device 102, patient programmer 120, and clinician programmer device 122. The therapy device 102 may be configured to communicate with patient programmer 120 and/or clinician programmer device 122 via network 124. In some implementations, the therapy device 102 is ex vivo or external to the body when applying therapy. In other implementations, the therapy device 102 is in vivo or internal to the body when applying therapy. In some such implementations, the therapy device 102 may be an implantable medical device (IMD).

The therapy device 102 may include a waveform generator, such as a sinusoidal waveform generator or a pulse generator 104 in the example of FIG. 1, a lead 106 (e.g., a stimulation and sensing lead), a controller 110, a memory 114, and a communication interface 118. The waveform generator (e.g., pulse generator 104) may include electronics, such as analog to digital converters (ADCs), digital to analog converters (DACs), filters, etc., configured to generate one or more electrical pulses in accordance with a set of stimulation parameters. The stimulation parameters may be configured by inputs or information, such as provided to therapy device 102 by patient programmer device 120, clinician programmer device 122, or both via network 124, to achieve a particular therapeutic effect when the one or more stimulation waveforms (also referred to herein as stimulation pulses, electrical pulses, stimulation therapy, etc.) are delivered to tissue of a patient. As described further herein, the stimulation parameters may be modified, such as adjusted, amplified, filtered, etc., to account for waveform distortion by using an inverse model of the tissue to be treated. The pulse generator 104 may be coupled to lead 106, controller 110, or both. In some implementations, the waveform generator is implanted or in vivo. For example, the pulse generator 104 includes or corresponds to an implantable pulse generator (IPG).

Lead 106 may be coupled to therapy device 102 to enable stimulation waveforms (e.g., the electrical pulses generated by the pulse generator 104) to be delivered to tissue of the patient via electrodes 108. Lead 106 includes a lead body having electrically conductive wires disposed therein. In some aspects, insulative material may surround the electrically conductive wires within the lead body of lead 106. Additionally, lead 106 may include an insulative or protective jacket surrounding the electrically conductive wires and the insulative material (if provided). The jacket may be formed from a biocompatible polymeric material, such as polyethylene, polypropylene, etc. to protect the lead wires and other components from fluids or other agents when lead 106 is implanted within the patient's body. In some aspects, lead jacket of lead 106 may include a plurality of openings through which one or more electrodes 108 may be exposed. In embodiments, lead 106 may be configured to be positioned within target anatomy of a patient, such as a region of the spine or another location.

Lead 106 may include a plurality of electrodes, such as electrodes 108. Electrodes 108 may include sensing electrodes, stimulation electrodes, sensing and stimulation electrodes, or combinations thereof. Generally, sensing electrodes may be configured to perform sensing operations, such as receiving or sensing signals (e.g., bioelectrical signals) generated by neural tissue of a patient, such as neuronal activity generated at the dorsal root ganglion of the patient. Stimulation electrodes may be configured to provide stimulation waveforms (such as pulses) to the neural tissue but may not be configured to perform sensing. Sensing and stimulation electrodes may be configured to both stimulate neural tissue and to sense neuronal activity associated with the stimulated neural tissue or adjacent neural tissue. In some aspects, electrodes 108 include sensing electrodes and stimulation electrodes, but not sensing and stimulation electrodes. In additional or alternative aspects, electrodes 108 may all be sensing and stimulation electrodes. In another additional or alternative aspect, electrodes 108 may include sensing and stimulation electrodes and stimulation electrodes. It is noted that while electrodes 108 have been described above as including specific arrangements or combinations of sensing, stimulation, and sensing and stimulation electrodes, such description has been provided for purposes of illustration, rather than by way of limitation and that other combinations and arrangements of electrodes and electrode types may be utilized by embodiments of the present disclosure.

Additionally, one or more of electrodes 108 may be directional electrodes configured to receive signals from a particular direction (e.g., from neurons positioned in a particular part of the neuroanatomy), to provide stimulation waveforms or pulses in a particular direction, such as towards neurons of a particular anatomical structure (e.g., neurons of the DRG, peripheral process, central process, or other issue of the patient), or both. Moreover, one or more of electrodes 108 may be omnidirectional electrodes, such as ring electrodes. It is to be understood, however, that the particular types, geometries, and/or configurations of the one or more electrodes 108 can be adapted based on exigencies of sensing and/or stimulating the neuroanatomy of interest.

Electrodes 108 may be spatially arranged along a lead body of lead 106 according to the target neuroanatomy of a patient, such as to provide electrodes configured to be proximate to the DRG, peripheral process, central process, and/or other neuroanatomy of the patient. For example, electrodes 108 adapted for use in closed loop techniques for treating patients via stimulation in accordance with the present disclosure may be grouped into two or more sets of electrodes, such as a first set of one or more electrodes and a second set of one or more electrodes, each set of electrodes targeting a specific target tissue of the patient (e.g., different portions the same tissue or different tissues). In an illustrative, non-limiting example, the electrodes or electrode sets may be configured to provide measurement pulses or waveforms and/or sense voltages during or caused by application of the measurement pulses or waveforms. Additionally, a third plurality (e.g., third set) of electrodes may be used in some aspects. For example, two sets of electrodes may be configured to provide measurement waveforms and therapy waveforms respectively, and a third set may be configured to sense voltages associated with the measurement waveform. It is noted that other arrangements of electrodes may also be utilized by embodiments, such as a single set of electrodes that includes sensing electrodes (e.g., for sensing neuronal signals, voltages, etc.) as well as stimulation electrodes.

Lead and/or electrode placement may be configured to reduce a quantity of noise (e.g., distortions in a neural signal, stimulation artifacts, etc.) observed by one or more of electrodes 108 (e.g., during sensing or recording of neuronal signals). The reduced noise provided via separating the different electrodes or sets of electrodes may enhance a signal to noise (SNR) ratio of electrodes 108 and enhance or improve the quality of neuronal signals recorded during sensing operations. For example, larger distances and/or different directions may reduce noise with respect to signals recorded or sensed by one or more of the electrodes 108 when the sensing is performed simultaneously with or subsequent to stimulation of patient tissue by other ones of the electrodes 108. In this manner, measurement data and/or feedback data generated by one or more electrodes may not be compromised or degraded by noise arising from a stimulation operation performed by an adjacent electrode or set of electrodes. It is to be understood that the exemplary therapy techniques disclosed herein for treating patient pain or other patient conditions may utilize a lead that includes more than two sets of electrodes or less than two sets of electrodes depending on the particular anatomy of interest, the waveform distortion for the tissue, the biomarkers or characteristics to be derived from the feedback data, or other factors.

Measurement data may include or correspond to data determined from or while applying a measurement signal or waveform. Such measurement data may include voltage or other measurements made by measuring or sensing signals applied to or generated by tissue during application of the measurement signal or waveform. Such measurement data may be associated with prefiltering or preadjusting a stimulation therapy prior to application of the therapy and may not require the therapy device or system 100 to have a closed loop or feedback system. Feedback data includes voltage or other measurements made during application of a stimulation waveform. In some implementations, feedback data may further include or encompass measurement data, such as measurements or feedback obtained from or during a measurement signal or waveform. As used herein, the term feedback data may be broadly used to encompass data or measurements obtained during application of measurement waveforms and stimulation waveforms. Measurement waveforms may differ from stimulation waveforms in some implementations. For example, a stimulation waveform may be a pulse type waveform and a measurement waveform may be a sinusoidal type waveform or non-current controlled waveform.

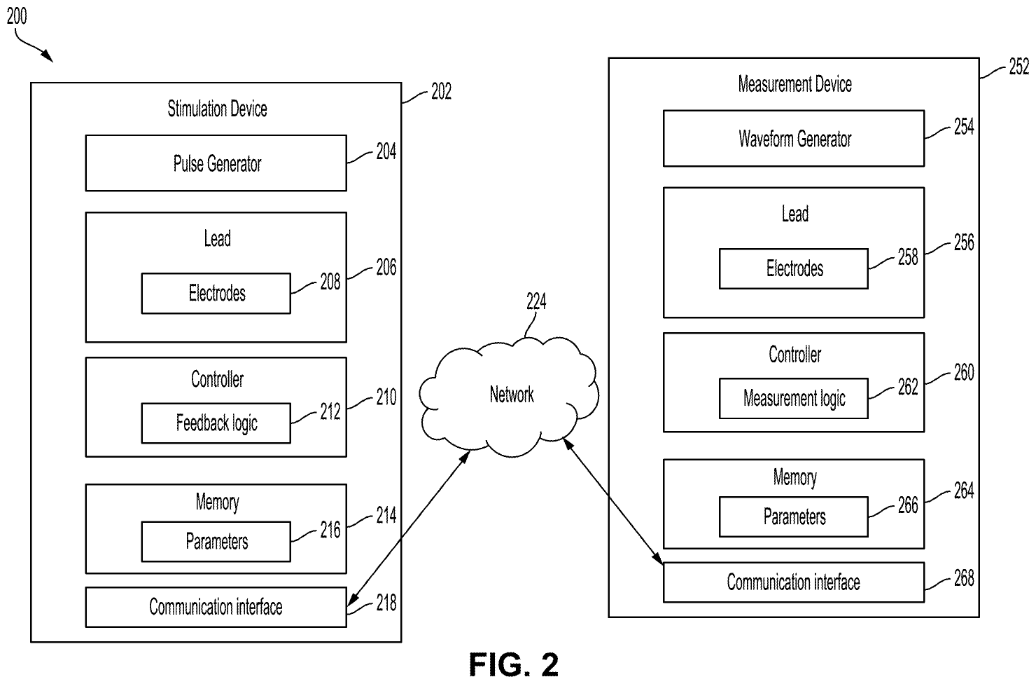

Controller 110 may be a microcontroller having one or more processors, one or more memories, and/or any of the foregoing. The one or more processors may include and/or correspond to one or more microprocessors, central processing units (CPUs), graphical processing units (GPUs), field programmable gate arrays (FPGAs), application specific integrated circuits (ASICs), and/or other logic circuitry configured to perform the operations of controller 110 described herein. Controller 110 may be communicatively coupled to lead 106, to one or more of electrodes 108, to memory 114, and/or to communication interface 118. Controller 110 may include compensation logic 112, such as measurement logic, feedback logic, etc. Compensation logic 112 may be circuitry, firmware, software, and/or any combination thereof configured to process measurement data, feedback data, or both, and to determine whether to modify one or more stimulation parameters (e.g., an amplitude, a frequency, a pulse width, a polarity, etc.) used to generate stimulation waveforms or pulses delivered to the target tissue of the patient. The measurement data and/or feedback data may be received from another device (as described further with reference to FIG. 2) or may be received from the one or more of electrodes 108, to analyze the measurement data and/or feedback data.

Additionally, controller 110 may be configured to control generation of stimulation waveforms by the waveform generator (e.g., stimulation pulses by the pulse generator 104) according to the stimulation parameters. In addition to managing generation and delivery of stimulation waveforms to select ones of electrodes 108, controller 110 may also be configured to control collection of feedback data corresponding to bioelectrical signals generated by neural tissue by particular ones of electrodes 108. In an aspect, the therapy device 102 may include one or more switches (not shown in FIG. 1) that may be configured by controller 110 to control delivery of stimulation waveforms to the target tissue of the patient by particular ones of electrodes 108 and to control recording or sensing of the feedback data by other ones of electrodes 108. For example, a stimulation switch may be configured to electrically couple different ones of electrodes 108 to the pulse generator 104 to enable stimulation pulses to be delivered to the target tissue and a sensing switch may be configured to electrically couple one or more of electrodes 108 to enable measurement data and/or feedback data to be collected and provided to compensation logic 112 for analysis.

The therapy device 102 may include memory 114. Memory 114 may include a random access memory (RAM), static RAM (SRAM), dynamic RAM (DRAM), static dynamic RAM (SDRAM), read only memory (ROM), programmable read only member (PROM), erasable programmable read only member (EPROM), electrically erasable programmable read only memory (EEPROM), optical storage, one or more hard disk drives (HDDs), solid state disk drives (SSDs), other memory devices configured to store data, instructions, or both in a persistent or a non-persistent state, or a combination of different memory devices. It is noted that while memory 114 is shown as a standalone component in FIG. 1, memory 114 may be distributed among various components of the therapy device 102. For example, a portion of memory 114 may be memory devices supporting operations of controller 110 (e.g., cache memory, etc.) or other components of therapy device 102.

Memory 114 may store instructions (e.g., software, firmware, etc.) and/or data. For example, memory 114 may include a non-transitory computer-readable storage medium having instructions that, when executed one or more processors (e.g., one or more processors of controller 110), cause the one or more processors to perform operations for providing closed loop stimulation therapy in accordance with aspects of the present disclosure. In an aspect, memory 114 may further be configured to store parameters 116 that include and/or correspond to stimulation parameters used to control operations of the therapy device 102, such as to control characteristics of the stimulation waveforms or pulses delivered to the patient (e.g., whether the waveforms or pulses are continuous or intermittent, a frequency, an amplitude, a pulse width, a polarity, or other parameters). It is noted that the stimulation parameters may be periodically changed or modified, either by operations of patient programmer 120 or clinician programmer 122, based on feedback data (e.g., inverse model information) received from electrodes 108, or combinations thereof.

In some aspects, parameters 116 may include active parameters (e.g., parameters that may be used to configure stimulation therapies for the patient), as well inactive parameters (e.g., parameters that were previously used to configure stimulation therapies for the patient but which are not being used presently). Storing active and inactive parameter sets at memory 114 may enable a user (e.g., a clinician or the patient) to view historical parameters and current parameters to evaluate aspects of the patient's treatment, such as to identify parameters that were inactivated as being ineffective in treating the patient, active parameters used to effectively treat the patient, or for other purposes. In some aspects, memory 114 may store other types of information, such as data corresponding to waveform distortion, such as due to tissue permittivity or capacitance, and/or data corresponding to one or more biomarkers associated with neuronal signals indicative of a state of the patient's condition (e.g., high pain, moderate pain, and/or low or no pain). As an example, the memory 114 may include model data, such as linear model data, forward model data, inverse model data, model parameter data, etc., corresponding to data used to measure and account for waveform distortion. Moreover, memory 114 may store data, such as statistical data, electrical data, etc., corresponding to neuronal activity indicative of high pain, moderate pain, and or low or no pain experienced by the patient.

Additionally, the therapy device 102 may include or be communicatively coupled to other components (not depicted in FIG. 1), such as one or more sensors (e.g., a voltage sensor, an accelerometer, a gyroscope, a heart rate sensor, a blood pressure sensor, a temperature sensor, galvanic skin conductance sensor, EEG sensor, and/or other types of sensors). As an example, controller 110 may receive sensor data from the one or more sensors, such as voltage data from a voltage sensor, accelerometer data from an accelerometer, indicating a mobility state of the patient (e.g., whether the patient is ambulatory, etc.), and the mobility state may provide an additional piece of information that indicates whether the patient is experiencing pain. Controller 110 may analyze the sensor data and the measurement and/or feedback data to determine a state or a condition of the patient (e.g., detecting waveform distortion, such as due to tissue permittivity or capacitance, or detecting biomarkers or other indications the patient is experiencing pain) and may adjust the stimulation parameters accordingly. For example, voltage measurements from a voltage sensor may be indicative of a model of the tissue and electrode, which can be manipulated to provide a model (and/or inverse model) of the tissue, which can then be used to adjust for waveform distortion, as further described with reference to FIGS. 8-10.

As briefly noted above, the therapy device 102 may be communicatively coupled to patient programmer device 120 and/or clinician programmer device 122 via the network(s) 124. To facilitate such communication, the therapy device 102 may include communication interface 118. Communication interface 118 may be a transceiver, a transmitter, a receiver, or any combination thereof. Additionally or alternatively, communication interface 118 may be networking hardware capable of communicating (e.g., receiving and/or sending data) with external devices using one or more communication standards or protocols (e.g., IEEE 802.11, Bluetooth™, Bluetooth Low Energy (BLE), Zigbee™, any of the 3G, 4G, or 5G communication protocols, other communication protocols, or combinations thereof), or other communication techniques.

Network 124 may include peer-to-peer networks, wireless fidelity (Wi-Fi) networks, wide area networks (WANs), local area network (LANs), the Internet, or other types of communication networks that may be utilized to facilitate the exchange of data between therapy device 102, patient programmer 120, and clinician programmer 122, or combinations thereof. In embodiments, various security measures and protocols (e.g., encryption, certificates, digital signatures, etc.) may be leveraged by system 100 to facilitate secure communication of data exchanged between and among therapy device 102, patient programmer 120, and clinician programmer 122 over network 124.

Patient programmer device 120 may be a device (e.g., smartphone, tablet computing device, laptop computing device, or another computing device) configured to provide instructions to and/or receive data from the therapy device 102. Patient programmer device 120 may be principally associated with the patient in whom the therapy device 102 is implanted. Clinician programmer 122 may be a device (e.g., smartphone, tablet computing device, laptop computing device, desktop computing device, or other types of computing devices) configured to provide functionality that enables a clinician to create and send instructions to the therapy device 102 and/or receive data from the therapy device 102. Clinician programmer 122 may be principally associated with a health care provider. In some aspects, patient programmer 120 and clinician programmer 122 may also be configured to exchange data via network(s) 124.

As briefly described above, the pulse generator 104 may be configured to generate stimulation pulses that may be delivered to target tissue of a patient to treat one or more medical conditions. In an exemplary mode of operation, the stimulation pulses generated by the pulse generator 104 may be delivered to spinal tissue of a patient via one or more electrodes among electrodes 108 to treat patient pain or another medical condition of the patient. In this example, the electrodes 108 may be implanted adjacent to the neural tissue of interest (e.g., a central process, a DRG, a peripheral process, etc.). The stimulation pulses may be generated by the pulse generator 104 based on stimulation parameters (e.g., parameters 116) configured to mitigate the treat the condition of the patient. For example, stimulation parameters used by the pulse generator 104 to generate the stimulation pulses may be configured to deliver stimulation pulses to tissue of the patient to block or attenuate transmission or relay of pain signals to the brain.

Delivery of stimulation waveforms or pulses to target neural tissue of the patient (e.g., the DRG) may block transmission of at least some pain signals, but a one-size-fits all or one-size-fits most approach may be insufficient to effectively treat a patient's chronic pain or other medical conditions. For example, clinician programmer 122 can be used during a session between the patient and a clinician to configure a set of stimulation parameters that effectively treats a specific level or intensity of pain for the patient, but the set of therapy configurations may be insufficient to treat pain experienced by the patient over time. As a result, the patient may experience discomfort (e.g., paresthesia caused by over stimulation of the target tissue) if the stimulation amplitude or frequency is too high, or may experience more intense pain if the pain increases above the level for which the stimulation parameters were intended (e.g., understimulation of the target tissue). Additionally, the pain experienced by the patient may vary according to a mobility state of the patient (e.g., is the patient lying down, sitting, standing, walking, running, and the like). As such, stimulation parameters for a pain management therapy intended for a particular level of pain may become insufficient due to changes in the patient's mobility state. Furthermore, therapy configurations suitable for one patient may be insufficient for another patient. One approach to address the challenges described above is to provide multiple sets of stimulation parameters, each configured for a different level or intensity of pain being experienced by the patient. While providing a greater degree of control over the therapies used to treat the patient's pain, these predetermined and static configurations still suffer from the same disadvantages described above (e.g., uncomfortable paresthesia due to overstimulation or intense pain due to under-stimulation) since the level of chronic pain experienced by the patient may not be well aligned with any specific one of the preconfigured stimulation parameters.

Closed loop neurostimulation systems offer a more dynamic approach to providing therapy, but the effectiveness of such systems is limited by the ability to reliably account for waveform distortion and modify the stimulation parameters accordingly. Treatment of chronic pain or other patient conditions using closed loop systems has been challenging due to difficulties accurately measuring and adjusting for waveform distortion caused by properties (e.g., permittivity, location, volume) of the tissue to be treated and even the frequency and/or amplitude of the treatment signal (simulation waveform or pulse). Below, exemplary aspects of closed loop systems and methods capable of addressing the above-described challenges are described.

As briefly described above, the therapy device 102 may be operated in a closed manner to provide therapy to a patient. In particular, controller 110 may be configured to receive feedback data from particular electrodes of electrodes 108. The feedback data may correspond to voltage measurements indicative of waveform distortion. Exemplary type of voltage measurements may include absolute voltage, differential voltage, voltage fluctuation over time, etc., or a combination thereof A plurality of voltage measurements may be obtained and may be associated with a corresponding measurement waveform and input data, such as input voltage, frequency, amps, etc. Such voltage measurements may be used to correct or compensate for waveform distortion as described further in detail below.

In addition to waveform distortion related feedback, the feedback data may include feedback data that may be used for other, non-waveform distortion related parameters or efficacy, such as for modifying stimulation waveforms and therapies to improve treatment of the patient (e.g., improve patient comfort with respect to pain mitigation therapies, etc.). As an illustrative example, the feedback data may correspond to neuronal signals (e.g., bioelectrical signals) indicative of pain (e.g., pain signals) as well as neuronal signals generated in response to delivery of the one or more stimulation waveforms or pulses to the target tissue of the patient. Exemplary types of neuronal signals that may be received as feedback data by controller 110 may include electroneurogram data corresponding to pain signals generated at neural tissue of the patient, ECAP data, neuronal data corresponding to neuronal activity of particular neurons of the neuroanatomy of the patient, such as neuronal activity of nociceptive neurons of a DRG of the patient, or other types of signals depending on the patient condition being treated and the corresponding patient anatomy. The above-described neuronal signals may be used to evaluate the pain state of a patient and adjust the therapy configuration for the patient based on the current pain state of the patient, which allows the therapy configuration to adapt to changes in the patient's pain state and maintain the patient's pain at comfortable levels.

Controller 110 may be configured to determine whether to modify the stimulation parameters based, at least in part, on the measurement data, the feedback data, or both. For example, waveform distortion of tissue can be estimated by measuring a voltage experienced by the tissue during application of stimulation waveform (such as for closed loop therapy) or during application of a dedicated measurement waveform (such as for prefiltered therapy). To further illustrate, compensation logic 112 of controller 110 may be configured to receive measurement or feedback data (e.g., voltage data) as an input and to determine, based on the measurement and/or feedback data, whether to modify one or more stimulation parameters. Illustrative examples of how to use the measurement and/or feedback data (e.g., voltage data) are described further with reference to FIGS. 8-11. As described in more detail below, system 100 may include various features that enable reliable detection of waveform distortion caused by tissue during application of a stimulation waveform and for use of the adjusted stimulation waveform in providing therapy to patients.

As briefly described above, compensation logic 112 of controller 110 may provide functionality for determining whether to modify or adjust stimulation parameters based on the measurement and/or feedback data. The functionality provided by compensation logic 112 may be configured to analyze the measurement and/or feedback data to enable waveform distortion adjustment, which may include detecting the presence of waveform distortion, such as waveform distortion caused by tissue permittivity, and modifying or adjusting the stimulation parameters when waveform distortion is detected. In an aspect, modification of the stimulation parameters may be configured to reduce waveform distortion or compensate for waveform distortion when applying the stimulation waveforms or pulses to the patient to provide therapy.

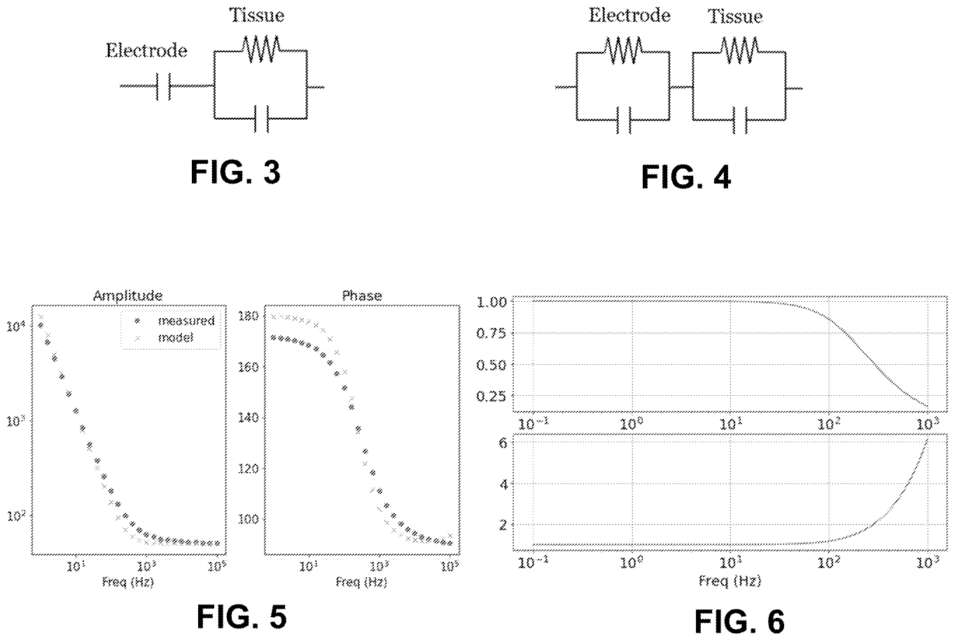

In response to a determination to adjust the stimulation parameters (e.g., based on the feedback data), controller 110 (or compensation logic 112) may be configured to modify one or more of the stimulation parameters to produce a modified set of stimulation parameters. For example, an amplitude, a frequency, and/or a phase of the one or more stimulation waveforms or pulses provided to target tissue may be adjusted based on an electrical model of the target tissue, which is determined based on voltage measurements. Example details for determination of the model of the target tissue are described further with reference to FIGS. 3-6.

Other stimulation parameters can be also adjusted to increase the therapeutic efficacy of neurostimulation including stimulation frequency and/or the temporal patterns of the stimulation waveforms. These other stimulation parameters may be configured to reduce waveform distortion in some implementations. For example, stimulation frequency and patterns of application of the waveforms may be adjusted based on the voltage measurements/feedback data. For example, a frequency of application of the waveform or pulses may be adjusted (e.g., reduced) based on voltage measurement fluctuations above a threshold. As another example, a frequency of the waveform itself may be adjusted (e.g., reduced) based due to voltage measurement fluctuations above a threshold.

Exemplary stimulation parameters that may be modified or adjusted include frequency, amplitude, pulse width, electrode configuration (e.g., selection of the anodes and cathodes used to deliver stimulation waveforms or pulses), burst pattern, and the like. To enhance the therapeutic benefit of electrical stimulation and reduce undesired side effects, including waveform distortion (and optionally stimulation induced discomfort), these parameters may be limited within certain boundary values and closed loop control techniques facilitate the automatic adjustment of these stimulation parameters within the configured boundary values. For example, a clinician may use a clinician programmer device to configure the boundary values (e.g., an upper and lower threshold for different stimulation parameters, such as frequency, amplitude, pulse width, and the like) during a programming session initiated between a therapy device and the clinician programming device. Once the boundary values are configured, adjustment of parameters in accordance with the closed loop techniques disclosed herein may be limited to parameter values within the boundary values configured by the clinician.

It is noted that in some instances the modification of the stimulation parameters may be configured to reduce a distortion effect caused by adjusting for pain experienced by a patient. For example, discomfort may be experienced by the patient when target tissue of the patient is overstimulated. This may occur when the stimulation parameters are configured for high levels of pain and the patient is experiencing low levels of pain. In such instances, controller 110 may modify the stimulation parameters to reduce the blocking effect to prevent the patient from experiencing uncomfortable paresthesia. When modifying the stimulation parameters to lower the blocking effect, controller 110 may ramp down the stimulation parameters gradually until a desired blocking effect is achieved. Additionally, the controller 110 may gradually adjust or compensate for changing waveform distortion experienced or exhibited by the tissue based on changes to the stimulation parameters used to treat the patient. In this manner, waveform distortion corrections can be made even while adjusting the therapy for other reasons, such as changes to alleviate symptoms of a condition of the patient (e.g., pain, symptoms of a movement disorder, etc.).

Additional stimulation waveforms or pulses configured based on the modified set of stimulation parameters may be subsequently delivered to the tissue of the patient via at least one electrode of the plurality of electrodes 108. For instance, controller 110 may cause the pulse generator 104 to generate stimulation pulses according to the modified set of stimulation parameters (based on the inverse model and/or to account for waveform distortion), and the stimulation pulses generated based on the modified set of stimulation parameters may be delivered to the target tissue of the patient, such as neural tissue of the patient, via the at least one electrode of the plurality of electrodes 108. It is noted that in some instances controller 110 (or compensation logic 112) may determine, based on the measurement and/or feedback data, not to modify the stimulation parameters (e.g., because the stimulation pulses are adequately compensating for waveform distortion, effectively treating a patient condition, or both).

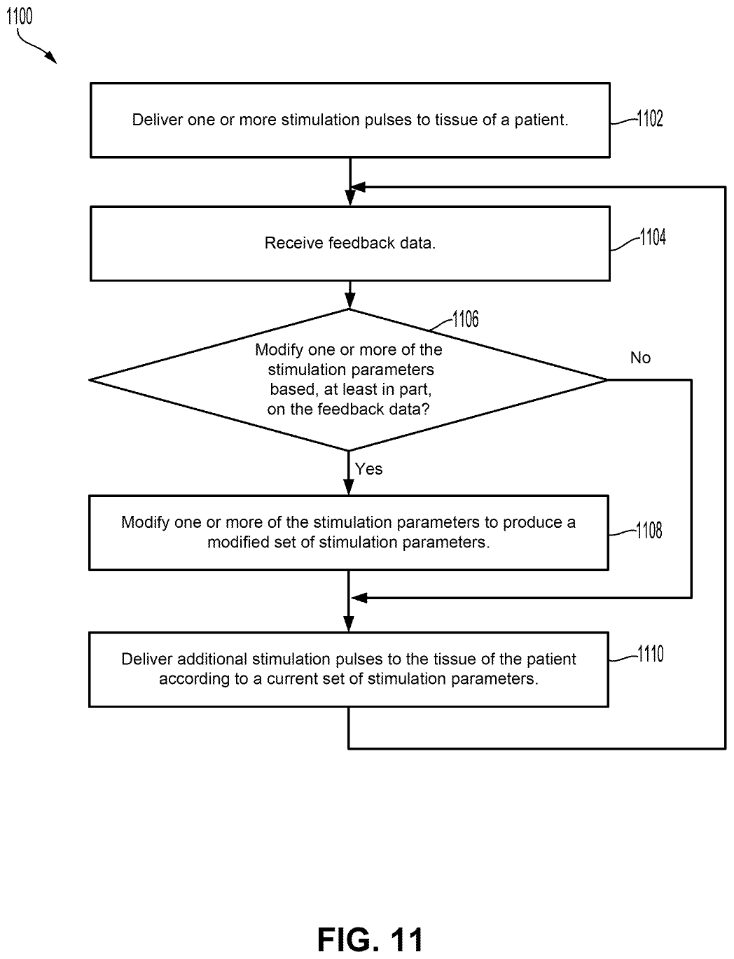

Closed loop electrical stimulation techniques in accordance with embodiments of the present disclosure may leverage different approaches with respect to adjusting for waveform distortion. Additionally, closed loop techniques for evaluating the patient condition state, detecting the presence of biomarkers indicative of symptoms of the patient condition, and enhancing an effectiveness of stimulation waveforms or pulses for treating the patient condition may be used with open or closed loop techniques for adjusting for waveform distortions. For example, a stimulation waveform may be prefiltered using a filter determined based on voltage measurements associated with application of a measurement waveform, and the feedback data may include neuronal data corresponding to neuronal activity of particular neural clusters in the neuroanatomy of the patient, such as neuronal activity of nociceptive neurons of the DRG of the patient. Accordingly, the system 100 can provide waveform distortion adjusted or corrected therapy to certain target tissue of a particular patient which compensates for the particular electrical characteristics of the tissue, location, volume, etc., and can further adjust the therapy (e.g., intensity and/or frequency) to adjust for patient comfort. When closed loop feedback (e.g., voltage measurements) are further used to adjust (e.g., refine) the stimulation parameters to correct for waveform distortion, the system 100 can further compensate for transient properties of the tissue, such as water content, oxygen content, fatigue, etc., which may cause fluctuations in tissue caused waveform distortion. An exemplary technique for using feedback data for closed loop electrical stimulation is described in more detail below with reference to FIG. 11. In such closed loop systems the feedback data may include voltage measurements (e.g., measurement data), neuronal data, or both.

Leveraging the above-described functionality enables system 100 to monitor and provide therapy that accounts for or reduces waveform distortion. To reliably reduce waveform distortion, system 100 incorporates one or more leads having electrodes configured to facilitate both stimulation of target neural tissue of the patient and recording of feedback data that may provide reliable information as to waveform distortion. The electrodes utilized by system 100 may be designed in a manner that mitigates noise and enhances the SNR of the electrodes utilized for sensing, while also providing a higher degree of directionality and control with respect to both sensing and delivery of stimulation waveforms or pulses. The enhanced capabilities of the electrodes utilized by system 100 enable reliable feedback to be recorded. The recorded feedback data may be analyzed by system 100 and used to determine whether to adjust one or more of parameters 116 (e.g., stimulation parameters). Modification of the one or more parameters 116 may be performed in certain situations based on the analysis of the feedback data, such as when modification of the one or more parameters will enhance an efficacy of waveform distortion reduction or compensation for target tissue of a particular patient. As shown above, the improved SNR and reduced noise provided by the configuration of the electrodes utilized by system 100 to sense or record the feedback data may enable various signals and waveform distortion to be reliably monitored by system 100. The ability to reliably observe the signals and waveform distortion provides a framework that may be used by system 100 to control a stimulation therapy to provide therapy which has reduced waveform distortion or provides therapy with waveform distortion adjusted waveforms.

Referring to FIG. 2, a block diagram illustrating aspects of another example therapy system according to embodiments of the present disclosure is shown as system 200. System 200 (e.g., a neurostimulation system) may be similar to system 100 of FIG. 1. For example, system 200 may be deployed to provide electrical stimulation, such as may be delivered to nervous system tissue of a patient to treat a condition or to spinal tissue of the patient to mitigate pain. The nervous system tissue may include brain tissue, neurons, axions, ganglions, nerves, etc. Additionally or alternatively, the therapy device may deliver therapy to other tissues, such as organ tissue, muscle tissue, etc.

As shown in FIG. 2, system 200 includes a stimulation device 202 and includes a measurement device 252 and/or a modeling device 272 communicatively coupled via a network 224. The components of FIG. 2 may include or correspond to the components of FIG. 1. For example, the components of FIG. 2 may have similar elements and functionality as compared to their counterparts of FIG. 1. To illustrate, the stimulation device 202, the measurement device 252, and/or the modeling device 272 of FIG. 2 may be similar to or be the same as the therapy device 102 of FIG. 1 (e.g., the stimulation device 202, the measurement device 252, and/or the modeling device 272 of FIG. 2 may have the one or more of the same elements and/or functionality as the therapy device 102 of FIG. 1). Similar to the system 100 of FIG. 1, system 200 may optionally include a patient programmer (e.g., the patient programmer 120 of FIG. 1), a clinician programmer device (e.g., the clinician programmer device 122 of FIG. 1), or both.

Although the three devices (the stimulation device 202, the measurement device 252, and the modeling device 272) are illustrated in the example of system 200 shown in FIG. 2, in other implementations the system 200 may include additional devices or fewer devices. For example, one or more of the three devices may be combined or integrated into a single device. To illustrate, the stimulation device 202 and the measurement device 252 may be combined or the measurement device 252 and the modeling device 272 may be combined. Additionally, or alternatively, system 200 may include multiple stimulation devices 202 and/or multiple measurement devices 252 which provide measurement and/or feedback information to the modeling device 272 for multiple tissue types, locations, and/or patients. In such implementations, the modeling device 272 may determine model information for use in programming the stimulation device 202 and/or measurement device 252, such as with multiple selectable models.

In the example of FIG. 2, the stimulation device 202 includes a waveform generator, such as a pulse generator 204, a lead 206 (e.g., a stimulation and/or sensing lead), a controller 210, a memory 214, and a communication interface 218. The waveform generator (pulse generator 204), the lead 206, the controller 210, the memory 214, and the communication interface 218, may include or correspond to the waveform/pulse generator 104, the lead 106, the controller 110, the memory 114, and the communication interface 118 of FIG. 1, respectively.

In some implementations, such as the example of FIG. 2, the stimulation device 202 includes feedback logic 212. The feedback logic 212 may be configured to determine one or more measurements, such as feedback measurements (optionally including voltage measurements), during application of a stimulation waveform (or adjusted stimulation waveform, such as a prefiltered stimulation waveform) generated by the waveform generator (e.g., pulse generator 204). The voltage measurements may be used to adjust or readjust for waveform distortion, as described with reference to the compensation logic 112 of FIG. 1. Alternatively, the feedback logic 212 may be configured to determine one or more measurements for pain mitigation, as described with reference to the compensation logic 112 of FIG. 1.

The measurement device 252 may include a waveform generator 254, such as sinusoidal waveform generator or a pulse generator, a lead 256 (e.g., a stimulation and sensing lead), a controller 260, a memory 264, and a communication interface 268. The waveform generator 254, the lead 256, the controller 260, the memory 264, and the communication interface 268 may include or correspond to the waveform/pulse generator 104, the lead 106, the controller 110, the memory 114, and the communication interface 118 of FIG. 1, respectively.

In the example of FIG. 2, the measurement device 252 includes measurement logic 262. The measurement logic 262 may be configured to determine one or more measurements, such as voltage measurements, during application of a measurement waveform generated by the waveform generator 254.

The measurement logic 262 may be configured to determine model information based on the measurements (e.g., measurement information). For example, the measurement logic 262 may be configured to determine linear model information of one or more electrodes 258 of the lead 256 and of the target tissue to be treated. Additionally, or alternatively, the measurement logic 262 may be configured to determine forward model information, inverse model information, or both. The forward and inverse model information may include or correspond to models of a forward transfer function of the target tissue itself and an inverse transfer function of the target tissue itself. The forward and inverse model information may be calculated based on the linear model information. For example, the forward model information may be calculated based on the linear model information and measurement information, and the inverse model information may be calculated based on the forward model information. Additional details regarding such calculations or determinations are described further with reference to FIGS. 3-10. The controller 260 (e.g., the measurement logic 262) may be configured to perform one or more operations of FIG. 1 to apply measurement waveforms and/or determine voltage measurements of tissue during application of the measurement waveforms. Additionally, or alternatively, the controller 260 (e.g., the measurement logic 262) may be configured to perform and/or one or more operations as described with reference to FIGS. 3-10 to determine the various model information, or components thereof, based on the voltage measurements.

During operation of the system 200, the stimulation device 202 may be implanted in a patient or be external to a patient. The measurement device 252 may be external to the stimulation device 202, the patient, or both, and may apply a measurement waveform to the target tissue. For example, the waveform generator 254 generates a measurement waveform which is applied by the electrodes 258 of lead 256 to the target tissue. The measurement device 252, such as via electrodes 258 (e.g., second electrodes) of lead 256, via another lead, or another device, measures voltages of the target tissue during and/or caused by the measurement waveform. The measured voltages may be stored in memory 264, such as parameter information of parameters 266.

In some implementations, the measurement device 252 may transmit the measurement information (e.g., voltage information) to the stimulation device 202 via the communication interface 268. In such implementations, the stimulation device 202 may generate the inverse model or a filter based on the inverse model based on the received measurement information from the measurement device 252, such as received at the communication interface 218 via the network 224.

In other implementations, the measurement device 252 determines the inverse model information based on the measurement information, such as by performing one or more operations described with reference to FIGS. 3-10, and transmits the inverse model information to the stimulation device 202. To illustrate, the measurement device 252 may determine parameters of the linear model based on the measurement information, determine forward model information based on the parameters of the linear model, and determine inverse model information based on the forward model information.

After obtaining the inverse model information, the stimulation device 202 may then apply therapy based on or using the inverse model information (e.g., the inverse model of the tissue). For example, the pulse generator 204 (or alternatively a waveform generator) generates an adjusted or filtered stimulation waveform which is applied via electrodes 208 of lead 206. As another example, the pulse generator 204 (or alternatively a waveform generator) generates a stimulation waveform which is filtered by another device or circuit (e.g., a filter) of the stimulation device 202 and then applied via electrodes 208 of lead 206. Accordingly, the system 200 may apply filtered stimulation therapy (e.g., prefiltered stimulation therapy) to the patient and with a reduced size or complexity stimulation device 202. This may enable implantable medical devices to use filtered stimulation waveforms for waveform distortion corrected therapy without the added size, complexity, and power usage associated with such filtering.

Optionally, the system 200 may further provide closed loop therapy in some implementations. For example the stimulation device 202 may provide closed loop feedback for accounting for waveform distortion, adapting a stimulation therapy for pain management (or another type of stimulation therapy), or both, similar to as described with reference to FIGS. 1 and 11.

As compared to system 100 of FIG. 1, the system 200 of FIG. 2 includes a measurement device 252 configured to enable modification or filtration (e.g., prefiltration) of the stimulation waveform and the measurement device 252 is separate from the stimulation device 202. For example, the measurement device 252 may determine an inverse model of the tissue to be treated and provide the inverse model to the stimulation device 202 for use in modifying or filtering (e.g. prefiltering) the stimulation waveform used to treat the tissue/apply the therapy. As compared to system 200 of FIG. 2, the system 100 of FIG. 1 may receive or retrieve the inverse model from a database, either locally or remotely, or may determine the inverse model itself based on its own measurements. For example, the pulse generator 104 of FIG. 1 may be configured to generate measurement waveforms and stimulation waveforms (e.g., modified or filtered stimulation waveforms). As another example, the pulse generator 104 of FIG. 1 may be configured to generate multiple types of stimulation waveforms, such as target stimulation waveforms and modified stimulation waveforms. The modified stimulation waveforms may be generated based on a closed-loop system or arrangements and represent stimulation waveforms that have been adjusted or modified (e.g., feedback filtered) based on an inverse model of the tissue. Thus, the system 100 of FIG. 1 incorporates the measurement device into a single device (the therapy device), and the system 200 of FIG. 2 takes a distributed approach where the stimulation device is separate from the measurement device. The distributed system 200 of FIG. 2 may be more suitable when IMD's are used and/or when the measurement waveform is of a different waveform type than the stimulation waveform. For example, the distributed system 200 of FIG. 2 may be more suitable when the measurement device uses sinusoidal waveforms and the therapy device uses pulses (pulse waveforms or rectangular waveforms, e.g., constant amplitude waveforms).

Alternatively, in some implementations the system 200 includes the modeling device 272. In some such implementations, the system 200 may include the modeling device 272 in addition to the measurement device 252. The modeling device 272 may be similar to measurement device 252 and may include similar elements to the measurement device 252. For example, the modeling device 272 may be configured to determine modeling information and/or adjusted stimulation parameters for stimulation waveforms with reduced distortion from measurements received the measurement device 252 as described with reference to FIG. 1. To illustrate, the modeling device 272 may include a controller (e.g., controller 260), a memory (e.g., memory 264) and a communication interface (e.g., communication interface 268). The controller and memory may include modeling logic and modeling parameters. Optionally, the modeling device 272 may not include a waveform generator or a lead (electrodes) in some implementations. In some aspects, the modeling device 272 may include or correspond to a patient programmer 120 or clinician programmer 122 of FIG. 1 and is configured to control the stimulation device 202, the measurement device 252, or both. The modeling device 272 may be proximate or local to stimulation device 202 and/or measurement device 252 in some implementations, such as connected via Bluetooth or a local network. In other implementations, the modeling device 272 may be remote from the stimulation device 202 and measurement device 252, such as connected via the Internet.

In some other such implementations, the system 200 may include the modeling device 272 in place of the measurement device 252. In such implementations without a measurement device, the stimulation device 202 may apply the measurement waveforms and determine voltage measurements associated with the measurement waveforms, and the modeling device 272 may be configured to receive the voltage measurements and generate the tissue model, such as described with reference to FIGS. 3-6. The modeling device 272 may be configured to determine model information (e.g., a forward or inverse model of the tissue) based on the received measurement information, and may optionally be configured to determine adjusted stimulation parameters based on the model information. In such implementations, the modeling device 272 transmits the model information or adjusted parameters to the stimulation device 202 for use in applying waveform distortion corrected therapy.

During operation of the system 200 in implementations where the system 200 includes a modeling device, the stimulation device 202 may be implanted in a patient or be external to a patient. The measurement device 252 and/or the modeling device 272 may be external to the stimulation device 202, the patient, or both.

In some implementations, the stimulation device 202 may apply a measurement waveform to target tissue of a patient. For example, the pulse generator 204 generates a measurement pulse which is applied by the electrodes 208 of lead 206 to the target tissue. The stimulation device 202, such as via electrodes 208 (e.g., first electrodes) of lead 206, via another lead, or another device, measures voltages of the target tissue during and/or caused by the measurement waveform. The measured voltages may be stored in memory 214, such as parameter information of parameters 216.

In some other implementations, the measurement device 252 may apply a measurement waveform to target tissue of a patient. For example, the waveform generator 254 generates a measurement waveform which is applied by the electrodes 258 of lead 256 to the target tissue. The measurement device 252, such as via electrodes 258 (e.g., second electrodes) of lead 256, via another lead, or another device, measures voltages of the target tissue during and/or caused by the measurement waveform. The measured voltages may be stored in memory 264, such as parameter information of parameters 266.

In such implementations, the stimulation device 202 or the measurement device 252 transmits the measurement information (e.g., voltage information) to the modeling device 272 via the network 224. The modeling device 272 may generate the inverse model, or a filter based on the inverse model, from the received measurement information. The modeling device 272 may transmit the model information to the stimulation device 202, which receives the model information at the communication interface 218 via the network 224.

After obtaining the model information, the stimulation device 202 may then apply therapy based on or using the received model information (e.g., the inverse model of the tissue). For example, the pulse generator 204 (or alternatively a waveform generator) generates an adjusted or filtered stimulation waveform which is applied via electrodes 208 of lead 206. As another example, the pulse generator 204 (or alternatively a waveform generator) generates a stimulation waveform which is filtered by another device or circuit (e.g., a filter) of the stimulation device 202 and then applied via electrodes 208 of lead 206. Alternatively, the modeling device 272 may be configured to determine adjusted stimulation parameters or a filter (e.g., filter parameters) based on the inverse model information and to transmit information for the adjusted stimulation parameters or the filter to the stimulation device 202.

In some implementations, the measurement and/or model information are generated based on feedback information from one or more first patients, and the stimulation therapy is applied to one or more second patients. For example, multiple stimulation or measurement devices may be used to determine a data set of voltage feedback for different tissue types and locations from multiple patients, and a modeling device may determine multiple models for the different combinations of tissue types and locations. One or more stimulation devices can then be programmed with or updated to receive filters or model information for the different combinations of tissue types and locations, such as using the modeling device 272 or one or more programming devices (e.g., 120 and/or 122). The stimulation devices can then apply open or closed loop waveform distortion corrected therapy to patients based on the model information or parameters and based on an input regarding tissue type, location, volume, etc. Accordingly, the system 200 can take a distributed form and may enable application of filtered stimulation therapy (e.g., prefiltered stimulation therapy) to the patient and with a reduced size or complexity stimulation device 202 which compensates for tissue induced waveform distortion.

Thus, as compared to system 100 of FIG. 1, system 200 illustrates and describes various configurations of distributed systems for providing waveform distortion compensation therapy. The examples of distributed systems in FIG. 2 enable reduced complexity and cost devices to be implanted, as compared to system 100 of FIG. 1, which can still provide stimulation therapy which corrects or adjusts for waveform distortion due to tissue electrical properties. For example, a pulse generation type stimulation device may be implanted which may have a single set of electrodes for providing stimulation pulses. The pulse generation device may not have additional electrodes, sensing circuitry, etc. to perform measurement and/or modeling operations, and thus may be smaller, have reduced power consumption, reduced costs, etc. The pulse generation device can provide stimulation pulses with modified parameters to account for pulse distortion caused by the tissue based on measurements and/or models generated by one or more outside devices (e.g., the measurement device 252, modeling device 272, etc.). As indicated above, the stimulation device 202 may receive information (e.g., voltage feedback information or model information) indicating adjusted stimulation parameters and provided filtered stimulation therapy. As another example, the stimulation device 202 may be preprogramed with multiple prefiltered stimulation waveforms generated based on measurements and modeling information previously collected from one or more other patients. These prestored waveforms may be generated based on tissue responses of another patient or based on data from tissue responses of a plurality of patients. Additionally, in some implementations, the measurements and models may be generated using more complex waveforms and sensing systems, such as sinusoidal waveform generators and/or discrete voltage sensing electrodes and circuitry. Accordingly, system 200 enables flexible implementations of waveform distortion correction for many different distributed configurations.

FIG. 3 is an example of a linear model to approximate an electrode and tissue. In the example of FIG. 3, the electrode of the therapy device is approximated as a capacitor and the tissue to be stimulated is approximated as a resistor arranged in parallel with a capacitor.

FIG. 4 is another example of a linear model to approximate the electrode and tissue. As compared to the linear model of FIG. 3, the linear model of FIG. 4 has a different model or approximation for the electrode. In the example of FIG. 4, the electrode of the therapy device is approximated as a resistor arranged in parallel with a capacitor and the tissue to be stimulated is approximated as a resistor arranged in parallel with a capacitor.

The electrodes represented in the models shown in FIGS. 3 and 4 may include or correspond to any of the electrodes describe above with reference to FIG. 1 or FIG. 2. The linear model of FIGS. 3 and 4 obey the superposition principle (at least generally over the intended therapy parameters) and output voltage and current are a linear function of input voltage and current. The linear model may be used to predict the stimulation waveform experienced by the tissue. The linear model represents electrical characteristics (e.g., a transfer function) of an electrode of the therapy device and tissue to be treated. In the linear model example of FIG. 3, the electrode and the tissue are coupled in series.

In the linear models of FIGS. 3 and 4, the capacitor in the electrode model/approximation may represent double layer capacitance and the resistor in the electrode model may represent Faradaic reaction. At low stimulation amplitudes, Faradaic reaction for the electrode may be low or negligible, and thus a capacitor model for electrode may be appropriate for certain stimulation waveforms (e.g., low stimulation amplitudes), as illustrated in FIG. 3.

Although two example linear models are illustrated in FIG. 3 and FIG. 4, other linear models may be used. The example linear models in FIG. 3 and FIG. 4 represent simplified or low-complexity linear models. In other implementations, high or higher complexity linear models may be used. Such higher complexity linear models, as compared to the linear models in FIG. 3 and FIG. 4, may have additional elements. For example, a linear model may include additional resistors, capacitors, or both, to approximate the tissue, the electrode, and/or other elements, and these additional components may be coupled in series or parallel. To illustrate, a second resistor may be coupled in series with the capacitor or in parallel with both the resistor and the capacitor. Additionally, or alternatively, the linear model may have other types of components, such as inductors.

The linear models may be determined by a therapy or measurement device. For example, a plurality of stored linear models may reside in a memory of the device and be associated with particular tissues or areas. The device may then retrieve a linear model based on the tissue or area to be treated, such as based on specific tissue permittivity/capacitance. Additionally, or alternatively, the linear model may be selected based on a frequency of the stimulation waveform. As another example, the device may compare measurements to multiple models and select a model with a best fit, or a best fit for a particular treatment range or waveform.

FIG. 5 is an illustration of an approximation of the model and the amplitude and phase data obtained from actual measurements. In FIG. 5, two graphs are depicted, a left graph depicting model and measurement values for magnitude and a right graph depicting model and measurement values for phase. In the left graph for magnitude, measured amplitudes are plotted for in a vertical axis against corresponding frequencies on a horizontal axis. Additionally, model values for the amplitude of the determined linear model of the tissue are also plotted. FIG. 5 illustrates that the amplitude/magnitude of the voltage measurements made during sensing have a nice fit or approximation to the amplitude/magnitude of the linear model of the electrode and tissue.

In the right graph for phase, measured phases are plotted for in a vertical axis against corresponding frequencies on a horizontal axis. Additionally, model values for the phase of the determined linear model of the tissue are also plotted. FIG. 5 illustrates that the phase of the voltage measurements made during sensing have a nice fit or approximation to the phase of the linear model of the electrode and tissue. The measurements and model estimates of FIG. 5 illustrate that the amplitude generally decreases with an increase in frequency, and that the phase changes with frequency.

In FIG. 5, the measurements were obtained by an electrochemical impedance spectroscopy (EIS) test in a phosphate buffered saline solution with platinum iridium (Pt/Ir) macroelectrode. The measurement waveform used was sinusoidal and voltage measurements were obtained for various frequencies during application of the measurement waveform. The magnitude and phase of the various frequencies were determined based on the measured voltages. In other implementations, other types of measurement or test signals can be used, as described further below.

Although FIG. 5 illustrates an example of an EIS test or model values being obtained by EIS measurements, in other implementations other measuring methods may be used. For example, other types of waves may be used, such as rectangular, triangle, square, sawtooth, noise, etc. Sinusoidal waves may not be as practical and/or suitable for an IPG. For example, the IPG may utilize a pulse wave similar to a stimulation wave (e.g., rectangular) and may have less frequency components for sensing/measuring. When using other types of waveforms, such as fixed amplitude waves, a duty cycle or pulse width may be adjusted to perform the measurements. For example, parameter estimation may be done with rectangular pulses with different pulse widths. The profile of the voltage for a constant current stimulus pulse is measured, and the measured data is used to estimate the model parameters of the linear model.

FIG. 6 illustrates exemplary voltages models for tissue. In FIG. 6, two graphs are depicted, a top graph depicting a forward model of voltage across the tissue for given current controlled stimulation waveform and a bottom graph depicting an inverse model of the voltage across the tissue for given current controlled stimulation waveform. The graphs illustrate amplitude on a vertical axis and frequency on a horizontal axis with frequencies increasing from left to right. The forward model illustrates the amplitude generally decreasing with an increase in frequency, and the inverse model illustrates the opposite, that the amplitude generally increases with an increase in frequency. The forward model of the voltage across the tissue for given current controlled stimulation waveform can be determined from the linear model of the electrode and the tissue and the measurements, such as the phase and magnitude data in FIG. 5 determined from the voltage measurements.

Prior to generation of the forward model, the linear model is obtained and the component values (e.g., values of the capacitors and/or resistors) of the linear model are obtained using the measurements. Because the focus is the voltage across the tissue for a given current controlled stimulation, the forward model is the voltage across the tissue over a given current application. Because the model is linear, the forward model can be easily obtained in the frequency domain by dividing the voltage across the tissue by the total transfer function of the linear model.

The inverse model of the voltage across the tissue for given current controlled stimulation waveform shown in the bottom graph can be determined based on the forward model (top graph). The inverse model G(s) is a mathematical inverse of the forward model F(s) in the frequency domain (i.e. G(s)=1/F(s)). The inverse model represents what the current stimulation waveform profile should be for a given desired voltage waveform. That is, the inverse model represent a modification (e.g., such as by filtering) to the stimulation waveform actually applied such that the stimulation waveform actually experienced by the tissue is matches or is close to the desired stimulation waveform to apply to the tissue.

The inverse model illustrated in FIG. 6 may correspond to a high pass filter or variant thereof where higher frequencies are attenuated to a lesser extent (or not at all) as compared to lower frequencies. In the inverse model illustrated in FIG. 6, lower frequencies are attenuated to a greater extent than higher frequencies.

In order to compensate for distortion caused by the capacitive property of the tissue, the stimulation waveform may be modified or filtered (prefiltered) with the inverse model of the tissue. Then, the voltage experienced by the target tissue will be close to the original stimulation waveform. Typically, the tissue permeability is high at low frequency, and the inverse filter will have higher amplitude at high frequency compared to low frequency.

The filter may include one or more adjustable components, one or more selectable paths, one or more adjustable paths, or a combination thereof. For example, the filter may include one or more adjustable resistors, adjustable capacitors, adjustable inductors, adjustable amplifiers, etc. As another example, the filter may include a number of different paths with different filtering properties (different transfer function responses) and the filter may be adjusted by selecting one or more paths, such as by chaining one or more path and/or switching between paths. As yet another example, the filter may include multiple path segments and/or adjustable connections in which multiple components may be selectively arranged to generate the inverse model.

As an alternative to filtering, the stimulation waveform may be amplified based on the inverse model. To illustrate, an amplifier may be used to amplify the stimulation waveform based on the inverse model, such as the transfer function of the inverse model, to amplify higher frequency signals to a greater extent than lower frequency signals.

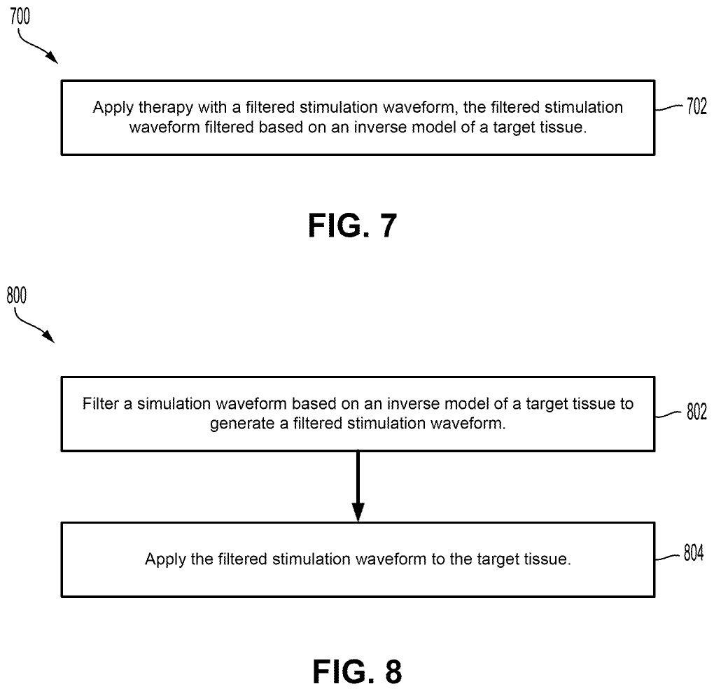

Referring to FIG. 7, a flowchart depicting an exemplary method for providing therapy which accounts for waveform distortion according to embodiments of the present disclosure is shown as a method 700. At block 702, method 700 includes applying therapy with a filtered stimulation waveform, the filtered stimulation waveform filtered based on an inverse model of a target tissue. For example, the therapy device 102 of FIG. 1 or the stimulation device 202 of FIG. 2 applies a stimulation waveform which has been modified by an inverse model of the target tissue, such as the inverse model of FIG. 6. Delivering one or more stimulation waveforms (e.g., stimulation pulses generated by an IPG, such as the pulse generator 104 of FIG. 1) to tissue of a patient via one or more electrodes (e.g., by one or more of electrodes 108 of FIG. 1 or one or more of electrodes 208 of FIG. 2).

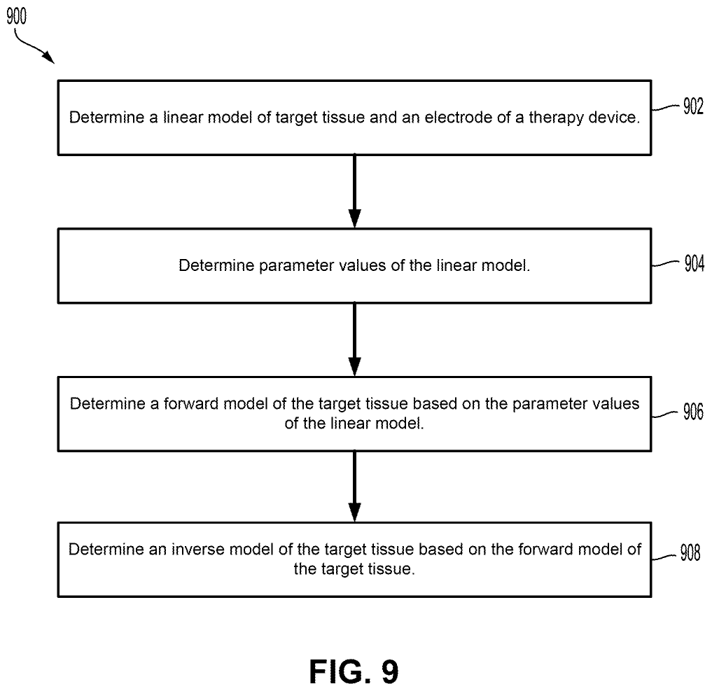

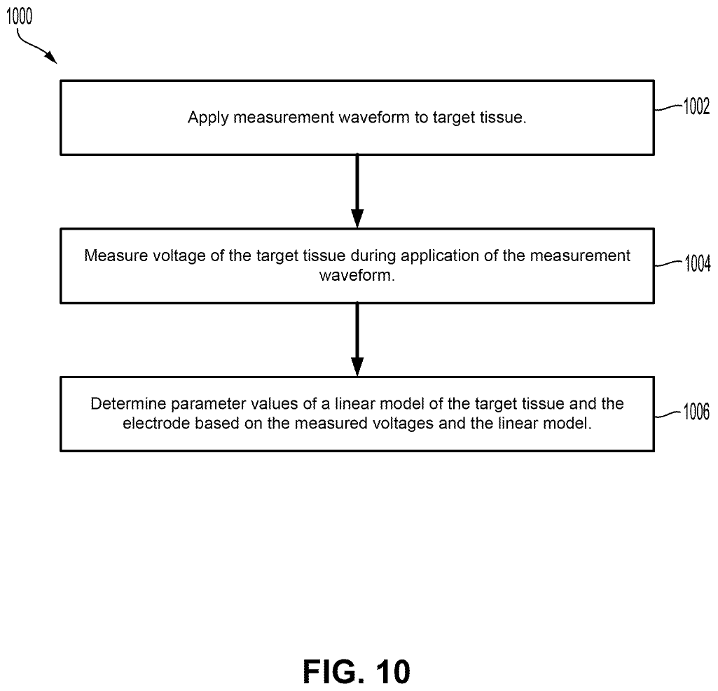

Referring to FIG. 8, a flowchart depicting an exemplary method for providing therapy which accounts for waveform distortion according to embodiments of the present disclosure is shown as a method 800. At block 802, method 800 includes filtering a simulation waveform based on an inverse model of a target tissue to generate a filtered stimulation waveform. For example, the therapy device 102 of FIG. 1 or the stimulation device 202 of FIG. 2 filters a desired stimulation waveform based on an inverse model, such as the inverse model of FIG. 6, of the target tissue to compensate for or reduce waveform disruption caused by tissue permittivity. To illustrate, the stimulation waveform may be filtered or modified such that the target tissue (e.g., neurons thereof) will actually experience the desired stimulation waveform or a stimulation waveform with reduced disruption from the desired stimulation waveform due to tissue permittivity. The inverse model of the target tissue may be determined as described with respect to FIGS. 1-7, or as described further with reference to FIGS. 9 and 10. Exemplary aspects of determining the inverse model of a target tissue are described in more detail below with reference to FIGS. 9 and 10.

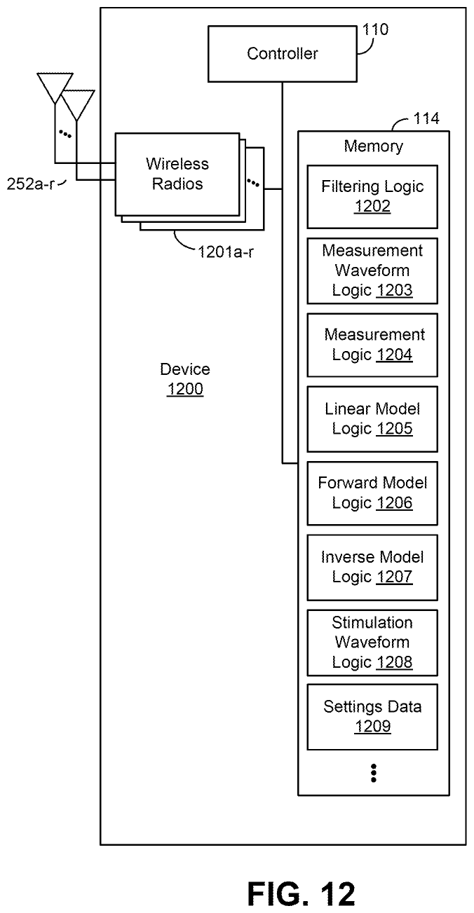

At block 804, method 800 includes applying therapy with a filtered stimulation waveform, the filtered stimulation waveform filtered based on an inverse model of a target tissue. For example, the therapy device 102 of FIG. 1 or the stimulation device 202 of FIG. 2 applies a stimulation waveform which has been modified by an inverse model of the target tissue, such as the inverse model of FIG. 6. Delivering one or more stimulation waveforms (e.g., stimulation pulses generated by an IPG, such as the pulse generator 104 of FIG. 1) to tissue of a patient via one or more electrodes (e.g., by one or more of electrodes 108 of FIG. 1 or one or more of electrodes 208 of FIG. 2). Exemplary aspects of controlling delivery of stimulation waveforms to target tissue of the patient and obtaining feedback data from the target tissue (or tissue proximate the target tissue) using different sets of electrodes are described in more detail below with reference to FIGS. 11 and 12.

Referring to FIG. 9, a flowchart depicting an exemplary method for determining an inverse model of tissue according to embodiments of the present disclosure is shown as a method 900. At block 902, method 900 includes determining a linear model of target tissue and an electrode of a therapy device. For example, the therapy device 102 of FIG. 1, the measurement device 252 of FIG. 2, and/or the modeling device 272 of FIG. 2 determines a circuit model of the tissue and the electrode which as a linear response, such as in linear models of FIGS. 3 and 4. As illustrated in the examples of FIGS. 3 and 4, the linear model may estimate or approximate the tissue and the electrode (e.g., one or more of electrodes 108 of FIG. 1 or one or more of electrodes 208 of FIG. 2) with circuit components (e.g., component with a linear response such as capacitors, resistors, inductors, etc.). Additionally, or alternatively, the linear model may have the tissue and the electrode coupled in series, as shown in the examples of FIGS. 3 and 4.

In some implementations, the linear model is selected based on the a type of the target tissue, such as brain tissue, spinal tissue, muscles tissue, etc. For example, the therapy device 102 of FIG. 1, the measurement device 252 of FIG. 2, and/or the modeling device 272 of FIG. 2 stores linear models associated with tissue types and selects a model based on the tissue type. The linear models may be generated in advanced based on experimentation.

Additionally, or alternatively, the linear model is selected based a target frequency of a stimulation waveform. For example, the therapy device 102 of FIG. 1, the measurement device 252 of FIG. 2, and/or the modeling device 272 of FIG. 2 stores linear models associated with different therapy frequencies or frequency ranges and selects a model based on the frequency of the stimulation. The linear models may be generated in advance based on experimentation. In addition, one or more other parameters may be used in addition to or in the alternative of the type of tissue and the frequency of the stimulation waveform, such as stimulation waveform power, stimulation waveform amplitude, stimulation waveform signal type (e.g., sinusoidal or square), volume of tissue, location of tissue, etc.

At block 904, method 900 includes determining parameter values of the linear model. For example, the therapy device 102 of FIG. 1, the measurement device 252 of FIG. 2, and/or the modeling device 272 of FIG. 2 determines parameter values of the linear response circuit model of the tissue and the electrode based on applying a measurement waveform to the target tissue to be stimulated and measuring or sensing voltage responses or voltage experienced by the target tissue. To illustrate, a measured voltages may be used to determine characteristics of the linear model (e.g., amplitude/magnitude and phase) and the characteristics of the measured model may be used to determine parameter values (e.g., resistance, capacitance, etc.) of the components (e.g., resistors, conductors, etc.) of the linear model. The parameter values of the linear model may be determined as described with respect to FIGS. 1-8, or as described further with reference to FIG. 10. Exemplary aspects of determining the parameter values of the linear model are described in more detail below with reference to FIG. 10.

In some implementation, the therapy device 102 of FIG. 1, the measurement device 252 of FIG. 2, and/or the modeling device 272 of FIG. 2 determine the parameter values. In other implementations, the measurement device 252 of FIG. 2 may send the measured voltages (or quantities derived therefrom, such as magnitude and phase) to the stimulation device 202 of FIG. 2 to use determining the parameter values. The measurement device 252 of FIG. 2 may then use the parameter values in providing therapy, such as by determining the forward and inverse models based on the parameter values.

At block 906, method 900 includes determining a forward model of the target tissue based on the parameter values of the linear model. For example, the therapy device 102 of FIG. 1, the measurement device 252 of FIG. 2, and/or the modeling device 272 of FIG. 2 determines a forward model of the tissue, such as the forward model of FIG. 6. As illustrated in the examples of FIGS. 3 and 4, the linear model may estimate or approximate the tissue and the electrode (e.g., one or more of electrodes 108 of FIG. 1 or one or more of electrodes 208 of FIG. 2) with circuit components (e.g., component with a linear response such as capacitors, resistors, inductors, etc.). The forward model may include or correspond to the voltage across the tissue over a given current injection/application. Additionally, or alternatively, the linear model may have the tissue and the electrode coupled in series, as shown in the examples of FIGS. 3 and 4.

In some implementation, the therapy device 102 of FIG. 1, the measurement device 252 of FIG. 2, and/or the modeling device 272 of FIG. 2 may calculate the forward model of the tissue. The measurement device 252 of FIG. 2 may then send the forward model to the stimulation device 202 of FIG. 2 to use in providing therapy, such as by filtering or prefiltering the stimulation waveform based on an inverse model thereof. In other implementations, the measurement device 252 of FIG. 2 determines the forward model and sends the forward model to the stimulation device 202 of FIG. 2, and the stimulation device 202 of FIG. 2 calculates the inverse model from the forward model.

Calculation of the linear model may include calculating the linear model in a frequency domain. For example, because the linear model has a linear response, the forward model can be obtained in the frequency domain by dividing the voltage measured across the tissue by the total transfer function of the linear model.

At block 908, method 900 includes determining an inverse model of the target tissue based on the forward mode of the target tissue. For example, the therapy device 102 of FIG. 1, the measurement device 252 of FIG. 2, and/or the modeling device 272 of FIG. 2 determines an inverse model of the tissue, such as the inverse model of FIG. 6, based on the forward model of the tissue. To illustrate, the therapy device 102 of FIG. 1 or the measurement device 252 of FIG. 2 may calculate the mathematical inverse of the forward model of the tissue to generate the inverse model of the tissue. The measurement device 252 of FIG. 2 may then send the inverse model to the stimulation device 202 of FIG. 2 to use in providing therapy, such as by filtering or prefiltering the stimulation waveform. In other implementations, the measurement device 252 of FIG. 2 determines the forward model and sends the forward model to the stimulation device 202 of FIG. 2, and the stimulation device 202 of FIG. 2 calculates the inverse model from the forward model. The inverse model G(s) may be a mathematical inverse of the forward model F(s) in the frequency domain (i.e. G(s)=1/F(s)).

Referring to FIG. 10, a flowchart depicting an exemplary method for determining parameter values of a linear model according to embodiments of the present disclosure is shown as a method 1000. At block 1002, method 1000 includes applying a measurement waveform to target tissue. For example, the therapy device 102 of FIG. 1 or the measurement device 252 of FIG. 2 delivers one or more measurement waves or pulses (e.g., measurement waves or pulses generated by an IPG, such as the pulse generator 104 of FIG. 1) to the target tissue of a patient via one or more electrodes (e.g., by one or more of electrodes 108 of FIG. 1 or one or more of electrodes 258 of FIG. 2).

In some implementations, the measurement waveform is a sinusoidal wave. For example, when an EIS device is used to generate and apply the measurement waveform, the measurement waveform may be a sinusoidal wave.

In other implementations, the measurement waveform is a pulse waveform, such as a rectangular pulse waveform. For example, when an implantable device is used to generate and apply the measurement waveform, the measurement waveform may be a pulse waveform similar to a pulse waveform of the stimulation therapy/waveform. In some such implementations, the pulse generator which is implanted in the patient has less complexity and/or components (e.g., reduced or no frequency components) to generate and measure the measurement pulse.

At block 1004, method 1000 includes measuring voltage of the target tissue during application of the measurement waveform. For example, the therapy device 102 of FIG. 1 or the measurement device 252 of FIG. 2 senses or measures a voltage of the tissue and induced or caused by the measurement waveform. To illustrate, the therapy device 102 of FIG. 1 or the measurement device 252 of FIG. 2 may use one or more leads or voltage sensors to determine a voltage or voltages of the tissue. A voltage or voltages may be sensed or measured multiple times for each measurement waveform. For example, the voltage or voltages may be measured at different frequencies of the measurement waveform, at different pulse widths of the measurement waveform, at different amplitudes/power, etc. or a combination thereof.

In some implementations, measuring the voltage further includes measuring and/or determining a magnitude (e.g., amplitude), a phase, or both, of the signal experienced by the tissue. For example, a magnitude and phase at each frequency of the measurement signal may be calculated by diving the measured voltage of the tissue by the input current of the measurement signal. This process may generate the response of the linear model of the electrode and the tissue as illustrated in the example of FIG. 5.

At block 1006, method 1000 includes determining parameter values of a linear model of the target tissue and the electrode based on the measured voltages and the linear model. For example, the therapy device 102 of FIG. 1, the stimulation device 202 of FIG. 2, the measurement device 252 of FIG. 2, and/or the modeling device 272 of FIG. 2 determines parameter values of the elements of the linear models. To illustrate, the therapy device 102 of FIG. 1, the stimulation device 202 of FIG. 2, the measurement device 252 of FIG. 2, and/or the modeling device 272 of FIG. 2 may calculate resistance of the resistors, capacitance of the capacitors, inductance of the inductors, etc., based on the measured voltages. In a particular implementation, the parameter values are estimated based on quantities derived from the measured voltages. The linear model of the electrode and tissue, with the estimated or determined parameters thereof, may be used to generate forward and inverse model of the tissue alone, as described above.

The parameter values may be calculated by any method. As an example, when the parameters are calculated using EIS, a low amplitude sinusoidal signal current is injected, and the voltage is measured as at 1004. The magnitude and phase at each frequency of the amplitude sinusoidal signal current is calculated by diving the measured voltage by the input current. Then the values of the resistors and capacitors are estimated to minimize the error between the linear model and the measured EIS data (measured phase and amplitude/magnitude).

As another example, the parameters may be calculated using rectangular pulses with different pulse widths. A voltage profile for the rectangular pulses is obtained in the measuring step at 1004, and the measured data is used to estimate the model parameters. For example, the values of the resistors and capacitors are estimated to minimize the error between the linear model and the voltage profile similar to estimating the parameter values with the EIS voltage data.

Referring to FIG. 11, a flowchart depicting an exemplary method for treating a patient using closed loop electrical stimulation according to embodiments of the present disclosure is shown as a method 1100. At block 1102, method 1100 includes delivering one or more stimulation waveforms (e.g., stimulation pulses generated by an IPG, such as the pulse generator 104 of FIG. 1) to tissue of a patient via a first set one or more electrodes (e.g., by one or more of electrodes 108 of FIG. 1 or one or more of electrodes 208 of FIG. 2).

At block 1104, method 1100 includes receiving feedback data from a second set of one or more electrodes (e.g., by one or more of electrodes 108 of FIG. 1 or one or more of electrodes 208 of FIG. 2). Exemplary aspects of controlling delivery of stimulation waveforms or pulses to target tissue of the patient and obtaining feedback data from the target tissue (or tissue proximate the target tissue) using different sets of electrodes are described in more detail below. At block 1106, method 1100 includes determining whether to modify one or more stimulation parameters (e.g., stimulation parameters used to generate the one or more stimulation waveforms or pulses delivered at 1102) based, at least in part, on the feedback data. As described herein, the modification of the stimulation parameters may be configured to enhance or improve an efficacy of the stimulation waveforms or pulses, such as to correct, reduce, or compensate for waveform distortion related effects. In addition to or in the alternative of waveform distortion related correction, non-related waveform distortion related modifications corrections can be achieved. For example, stimulation parameters may be modified to mitigate pain of the patient without the discomfort caused by overstimulation or the loss of the therapeutic efficacy due to understimulation (e.g., common problems of prior stimulation systems for treating pain of a patient).

It is noted that the determination to modify (or not modify) the stimulation parameters and the particular feedback considered, at block 1106, may depend on waveform distortion measurements (e.g., voltage measurements), the biomarker(s) utilized to identify a pain state or level of the patient, or a combination thereof. Exemplary techniques for determining whether to modify the one or more stimulation waveforms or pulses based on feedback data are described in more detail below with reference to FIGS. 8-10.

At block 1108, method 1100 includes modifying the one or more stimulation parameters to produce a modified set of stimulation parameters in response to determining to modify the one or more stimulation parameters. It is noted that in some aspects block 1108 may instead determine not to modify the stimulation parameters (e.g., if the feedback data and waveform distortion experienced by the tissue is adequately being accounted for by a current set of stimulation parameters).

At block 1110, method 1100 includes delivering additional stimulation waveforms or pulses to the tissue of the patient via at least one electrode of the plurality of electrodes according to a current set of stimulation parameters. It is noted that the current set of stimulation parameters may include the modified set of stimulation parameters or the set of stimulation parameters used to generate the stimulation waveforms or pulses delivered at block 1102 depending on whether the determining, at block 1106, indicates that the waveform is experiencing distortion above a threshold amount, that the patient's pain is adequately being treated or inadequately being treated, or both. For example, if, at block 1106, it is determined not to modify the one or more of the stimulation parameters, method 1100 may proceed to block 1110 and additional stimulation waveforms or pulses may be delivered to the neural tissue of the patient via the first set of one or more electrodes. After block 1110, method 1100 may return to 1104 at which feedback data again may be received from one or more electrodes.

To elaborate and place method 1100 in the context of FIGS. 1 and 2, at block 1102, a controller (e.g., controller 110 of FIG. 1) of a therapy device (e.g., therapy device 102 of FIG. 1, such as an IMD) may be configured to cause an IPG (e.g., the pulse generator 104 of FIG. 1, such as an IPG) to generate one or more stimulation waveforms or pulses according to stimulation parameters (e.g., parameters 116 of FIG. 1). The stimulation waveforms or pulses may be delivered to neural tissue of a patient via one or more electrodes (e.g., electrodes 108 of FIGS. 1 and 2). At block 1104, the controller may be configured to receive feedback data. As described herein, the feedback data may be received from a second set of one or more electrodes, which may be the same as or different from the first set one or more electrodes used to deliver the one or more stimulation waveforms or pulses at block 1102. The feedback data may correspond to voltage measurements used for waveform distortion correction as described with reference to FIGS. 1 and 2.

Alternatively or additionally, the feedback data may correspond to data used to improve or adjust non-waveform distortion related effects, as described with reference to FIGS. 1 and 2. For example, the feedback data may include neuronal signals generated in response to being stimulated by the stimulation waveforms or pulses before, during, or after stimulation. As another example, the feedback data may correspond to neuronal signals collected from neural tissue that has not been exposed to stimulation waveforms or pulses, such as neuronal signals associated with patient pain.

At block 1106, feedback logic (e.g., feedback logic of compensation logic 112 of FIG. 1) of the controller may be configured to analyze the feedback data to determine whether to modify the one or more stimulation parameters. For instance, the feedback logic may be configured to assess an amount of waveform distortion for the stimulation waveform experienced by the target tissue based, at least in part, on the feedback data. Exemplary techniques are described below for analyzing feedback data to assess the distortion and/or effectiveness of the stimulation waveforms or pulses.

In addition to performing waveform distortion correction, the feedback logic may be configured to assess an effectiveness of the stimulation waveforms or pulses in blocking generation and/or transmission of pain signals at nerve tissue of the patient based, at least in part, on the feedback data. Exemplary techniques are described below for analyzing feedback data to assess the effectiveness of the stimulation waveforms or pulses with respect to blocking the generation and/or transmission of pain signals at nerve tissue of the patient.

At block 1108, in response to determining to modify one or more of the stimulation parameters, the controller may be configured to modify the one or more stimulation parameters to produce a modified set of stimulation parameters. The modified set of stimulation parameters may be configured to correct for or reduce waveform distortion, such as by reducing waveform distortion caused by tissue based on an inverse model of the tissue, which is obtained from voltage measurement feedback. Additionally, or alternatively, the modified set of stimulation parameters may be configured to enhance an effectiveness of stimulation waveforms or pulses in attenuating generation and/or transmission of pain signals emanating from neural tissue of the patient, such as attenuating signals transmitted from the DRG toward the dorsal root or rootlets of the patient.

After the determination to modify one or more of the stimulation parameters at block 1108, the controller, at block 1110, may be configured to deliver additional stimulation waveforms or pulses to the tissue (or other neural tissue) of the patient via at least one electrode of the plurality of electrodes according to the modified set of stimulation parameters. Thenceforth, the controller may be configured to cause feedback data (e.g., second feedback data) to again be received from a second set of one or more electrodes for the purpose of evaluating an effectiveness of the modified set of stimulation parameters to correct for or reduce waveform distortion. In some implementations, where the closed loop feedback is used for pain control, the second feedback data may be used to evaluate the effectiveness of the modified set of stimulation parameters at blocking pain signal generation and/or propagation (e.g., minimizing or mitigating the patient's perception of pain).

However, if, at block 1106, the controller determines not to modify the one or more of the stimulation parameters, the controller may be configured to cause a pulse generator to deliver additional stimulation pulses to the tissue of the patient via at least one electrode of the plurality of electrodes according to the unmodified stimulation parameters. Thereafter, the controller may be configured to cause feedback data to again be received from a second set of one or more electrodes for the purpose of re-evaluating an effectiveness of the stimulation parameters at correcting for or reducing waveform distortion. Alternatively, in lieu of causing a pulse generator to deliver additional stimulation pulses to the target tissue of the patient, the controller may be configured to temporarily cease delivery of additional stimulation waveforms or pulses unless feedback data is received at the controller indicating waveform distortion decreasing, not increasing, or below a threshold. For example, in response to receipt of feedback data indicating increased waveform distortion, the controller may temporarily cease delivery of stimulation waveforms or pulses to the target tissue. Another type of stimulation waveform (e.g., different stimulation parameters) may be used or a different placement of electrodes may be used to resume therapy.

As shown above, FIG. 11 provides an overview of a closed loop stimulation algorithm for providing therapy to a patient with a distortion corrected stimulation waveform in accordance with aspects of the present disclosure. Method 1100 may be particularly well suited for applications involving a wide range of tissue types and target areas. In particular, a device may be able to compensate for waveform distortion of different tissue types and/or for different locations or volumes of the same tissue type. As a result, neurostimulation systems operating in accordance with the present disclosure, whether according to the method 1100 or one of the other methods disclosed herein, may provide improved capabilities to capture feedback for use as inputs to closed loop stimulation algorithms and control processes and provide improved closed loop control of waveform distortion corrected stimulation therapies utilized to treat patients. It should be understood that the closed loop stimulation algorithms disclosed herein are not limited to method 1100.

Steps of the various methods described above, such as method 700 to method 1100 may be stored as instructions in a memory of the a therapy device as described with reference to FIGS. 1 and 2. For example, one or more steps of any of method 700 to method 1100 may be stored as instructions in a memory (e.g., the memory 114 of FIG. 1) of the therapy device 102 of FIG. 1. The instructions may be executable by one or more processors (e.g., processors of the therapy device 102, the controller 110 of FIG. 1, or other logic) to perform closed loop stimulation in connection with compensating for waveform distortion (and/or managing a patient's pain) in accordance with the concepts disclosed herein.

FIG. 12 is a block diagram illustrating a device 1200 configured according to one aspect of the present disclosure. The device 1200 may include the structure, hardware, and components as illustrated for any of the devices of FIG. 1 or 2. For example, device 1200 may include or correspond to the therapy device 102 of FIG. 1, the stimulation device 202 of FIG. 2, the measurement device 252 of FIG. 2, or the modeling device 272 of FIG. 2. In the example, of FIG. 12, device 1200 includes a controller/processor 110 which operates to execute logic or computer instructions stored in memory 114, as well as controlling the components of device 1200 that provide the features and functionality of device 1200. Device 1200, under control of controller/processor 110, transmits and receives signals via wireless radios 1201a-r and antennas 252a-r. Wireless radios 1201a-r include various components and hardware including modulator/demodulators, a receive processor, a transmit processor, etc. As illustrated in the example of FIG. 12, memory 114 stores filtering logic 1202, measurement waveform logic 1203, measurement logic 1204, linear model logic 1205, forward model logic 1206, inverse model logic 1207, and simulation waveform logic 1208. The memory 114 may also store settings data 1209. The settings data 1209 may include or correspond to waveform data, measurement data, model data, model parameter data, or a combination thereof. The waveform data may include data for measurement and/or stimulation waveforms. The model data may include model data for one or more linear models, forward models, inverse models, or a combination thereof.

The device 1200 may perform any of the various methods described above, such as method 700, method 800, method 900, method 1000, or method 1100. For example, the device 1200 may perform one or more steps of any of the methods from method 700 to method 1100. To illustrate, operations may be stored as instructions in the memory 114 and carried out or performed by the corresponding logic (1202-1208) of device 1200. For example, the filtering logic 1202 may include instructions and/or data for performing filtering operations as described above, such as filtering a stimulation waveform to generate a filtered stimulation waveform. As another example, the waveform logics (1203 and 1208) may be configured to generate and apply the respective measurement and stimulation waveforms. As another example, the measurement logic 1204 may be configured to perform measurement operations, such as one or more of those described with reference to FIGS. 9 and 10. To illustrate, the measurement logic 1204 may be configured to control or perform the sensing or measuring or voltages and/or the adjustment of the measurement waveform. As yet another example, the various model logics (1205-1207) may be configured to determine, select, generate or otherwise obtain the respective models of the linear model, the forward model, and the inverse model. To illustrate, the linear model logic 1205 may obtain the linear model as described with reference to FIG. 9, the forward model logic 1206 may obtain the forward model from the linear model and measurements as described with reference to FIGS. 9 and 10, and the inverse model logic 1207 may obtain the inverse model from the forward model as described with reference to FIG. 9.

Additionally, it is noted that the exemplary techniques of FIGS. 8-11 may be used individually, or in combination. For example, the determining parameter values of a linear model of the method 1000 of FIG. 10 may be performed to determine the inverse model using the method 900 of FIG. 9. As another example, the close loop feedback technique of FIG. 11 may be used with one or more of the methods of FIGS. 8-10. To illustrate, conventional closed loop feedback may be used to improve stimulation therapy with a prefiltered stimulation wave that is prefiltered based on the inverse model. This may further reduce waveform distortion and improve efficacy of the treatment.

Furthermore, it is noted that when adjusting the stimulation parameters based on the measurement and/or feedback data, the therapy device operating in accordance with the concepts disclosed herein may be configured to make multiple adjustments to the stimulation parameters and monitor feedback data to evaluate how effective each adjustment is at providing therapy (e.g., mitigating patient pain). For example, stimulation waveforms or pulses having different amplitudes may be delivered sequentially upon making a determination to modify the stimulation parameters and feedback data may be obtained for each different amplitude. The amplitude associated with feedback data indicating a higher effectiveness at blocking patient pain perception may be selected for use in treating the patient's pain. Subsequently, situations may arise where the parameter(s) selected as providing the best improvement to the patient's pain may no longer be effective (e.g., based on analysis of the feedback data using the techniques described above), such as due to a different state of the patient (e.g., running, walking, standing, laying down, etc., or other reasons), and a different set of adjustments may be made to the stimulation parameters. Furthermore, it is noted that while the non-limiting example described immediately above involves titrating different amplitude levels and then selecting one for use in treating the patient's pain, any stimulation parameters may be similarly adjusted and evaluated, individually or in combination with other parameters, to identify a set of stimulation parameters providing improved mitigation of patient pain in accordance with the concepts disclosed herein. Accordingly, therapy devices and/or systems may provide waveform distortion compensated therapy by determining and utilizing an inverse model of the tissue to be treated to filter a stimulation waveform. By utilizing stimulation waveforms filtered with inverse tissue models treatment efficacy may be increased by reducing the amount of waveform distortion experienced by the tissue.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Components, the functional blocks, and the modules described herein with respect to FIGS. 1-12) include processors, electronics devices, hardware devices, electronics components, logical circuits, memories, software codes, firmware codes, among other examples, or any combination thereof. In addition, features discussed herein may be implemented via specialized processor circuitry, via executable instructions, or combinations thereof.

Those of skill would further appreciate that the various illustrative logical blocks, modules, circuits, and algorithm steps described in connection with the disclosure herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Skilled artisans will also readily recognize that the order or combination of components, methods, or interactions that are described herein are merely examples and that the components, methods, or interactions of the various aspects of the present disclosure may be combined or performed in ways other than those illustrated and described herein.

The various illustrative logics, logical blocks, modules, circuits, and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules, and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or any conventional processor, controller, microcontroller, or state machine. In some implementations, a processor may also be implemented as a combination of computing devices, such as a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or any combination thereof. Implementations of the subject matter described in this specification also may be implemented as one or more computer programs, that is one or more modules of computer program instructions, encoded on a computer storage media for execution by, or to control the operation of, data processing apparatus.

If implemented in software, the functions may be stored on or transmitted over as one or more instructions or code on a computer-readable medium. The processes of a method or algorithm disclosed herein may be implemented in a processor-executable software module which may reside on a computer-readable medium. Computer-readable media includes both computer storage media and communication media including any medium that may be enabled to transfer a computer program from one place to another. A storage media may be any available media that may be accessed by a computer. By way of example, and not limitation, such computer-readable media can include random-access memory (RAM), read-only memory (ROM), electrically erasable programmable read-only memory (EEPROM), CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, hard disk, solid state disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those skilled in the art, and the generic principles defined herein may be applied to some other implementations without departing from the spirit or scope of this disclosure. Thus, the claims are not intended to be limited to the implementations shown herein, but are to be accorded the widest scope consistent with this disclosure, the principles and the novel features disclosed herein.

Additionally, a person having ordinary skill in the art will readily appreciate, the terms “upper” and “lower” are sometimes used for ease of describing the figures, and indicate relative positions corresponding to the orientation of the figure on a properly oriented page, and may not reflect the proper orientation of any device as implemented.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a sub combination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted may be incorporated in the example processes that are schematically illustrated. For example, one or more additional operations may be performed before, after, simultaneously, or between any of the illustrated operations. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, some other implementations are within the scope of the following claims. In some cases, the actions recited in the claims may be performed in a different order and still achieve desirable results.

As used herein, including in the claims, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically; two items that are “coupled” may be unitary with each other. the term “or,” when used in a list of two or more items, means that any one of the listed items may be employed by itself, or any combination of two or more of the listed items may be employed. For example, if a composition is described as containing components A, B, or C, the composition may contain A alone; B alone; C alone; A and B in combination; A and C in combination; B and C in combination; or A, B, and C in combination. Also, as used herein, including in the claims, “or” as used in a list of items prefaced by “at least one of” indicates a disjunctive list such that, for example, a list of “at least one of A, B, or C” means A or B or C or AB or AC or BC or ABC (that is A and B and C) or any of these in any combination thereof. The term “substantially” is defined as largely but not necessarily wholly what is specified—and includes what is specified; e.g., substantially 90 degrees includes 90 degrees and substantially parallel includes parallel—as understood by a person of ordinary skill in the art. In any disclosed aspect, the term “substantially” may be substituted with “within [a percentage] of” what is specified, where the percentage includes 1, 5, and 10 percent; and the term “approximately” may be substituted with “within 10 percent of” what is specified. The phrase “and/or” means and or.

Although the aspects of the present disclosure and their advantages have been described in detail, it should be understood that various changes, substitutions and alterations can be made herein without departing from the spirit of the disclosure as defined by the appended claims. Moreover, the scope of the present application is not intended to be limited to the particular implementations of the process, machine, manufacture, composition of matter, means, methods and processes described in the specification. As one of ordinary skill in the art will readily appreciate from the present disclosure, processes, machines, manufacture, compositions of matter, means, methods, or operations, presently existing or later to be developed that perform substantially the same function or achieve substantially the same result as the corresponding aspects described herein may be utilized according to the present disclosure. Accordingly, the appended claims are intended to include within their scope such processes, machines, manufacture, compositions of matter, means, methods, or operations.

What is claimed is:

1. A method of providing stimulation therapy to a patient, the method comprising:
filtering a simulation waveform based on an inverse model of a target tissue to generate a filtered stimulation waveform; and
applying the filtered stimulation waveform to the target tissue.

2. The method of claim 1, wherein the stimulation waveform corresponds to a desired therapy waveform, and wherein the filtered stimulation waveform is configured to reduce waveform distortion caused by the target tissue as compared to the stimulation waveform.

3. The method of claim 1, wherein filtering the simulation waveform based on the inverse model includes:
determining a filter based on the inverse model; and
applying the filter to the simulation waveform, wherein the filter attenuates lower frequencies to a greater extent than higher frequencies.

4. The method of claim 1, wherein filtering the simulation waveform based on the inverse model includes:
determining an amplifier or amplifier settings based on the inverse model; and
modifying or amplifying to the simulation waveform based on the amplifier or amplifier settings.

5. The method of claim 1, further comprising:
determining the inverse model of the target tissue based on a linear model of the target tissue and an electrode used to apply the stimulation waveform and based on measured voltages of the target tissue caused by application of a measurement waveform.

6. The method of claim 5, wherein determining the inverse model of the target tissue further includes:
determining parameter values of the linear model based on the linear model and the measured voltages;
determining a forward model of the target tissue based on the linear model and the parameter values of the linear model; and
determining the inverse model of the target tissue based on the forward model of the target tissue.

7. The method of claim 5, wherein determining the inverse model of the target tissue further includes:
determining the linear model of the target tissue and the electrode based on the target tissue, the stimulation waveform, or both.

8. The method of claim 7, wherein determining the linear model based on the target tissue, the stimulation waveform, or both includes:
determining the linear model based a type or capacitance of the target tissue and based on a target frequency or amplitude of the stimulation waveform.

9. The method of claim 5, wherein the linear model is configured to model electrical properties of the electrode and the target tissue, wherein the electrode and the target tissue are coupled in series in the linear model, and wherein the electrode is approximated as a capacitor and the target tissue is approximated as a resistor and capacitor in parallel.

10. The method of claim 5, wherein the linear model is configured to model electrical properties of the electrode and the target tissue, wherein the electrode and the target tissue are coupled in series in the linear model, and wherein the electrode is approximated as a capacitor and a resistor in parallel and the target tissue is approximated as a resistor and a capacitor in parallel.

11. The method of claim 6, wherein determining the parameter values of the linear model based on the linear model and the measured voltages includes:
determining the parameter values of the linear model using an electrochemical impedance spectroscopy (EIS) device.

12. The method of claim 11, wherein determining the linear model using the EIS device includes:
applying a sinusoidal signal to the target tissue;
measuring a voltage of the applied sinusoidal signal experienced by the target tissue for each frequency of the applied sinusoidal signal;
determining a magnitude and a phase for each frequency of the applied sinusoidal signal based on the measured voltage and the applied sinusoidal signal; and
determining the parameter values for the linear model based on the magnitude and the phase and based on the linear model.

13. The method of claim 6, wherein determining the parameter values of the linear model based on the linear model and the measured voltages includes:
determining the parameter values of the linear model using a rectangular pulse generator.

14. The method of claim 13, wherein determining the linear model using the rectangular pulse generator includes:
applying multiple rectangular pulse waves of a constant current and different pulse widths to the target tissue;
measuring voltages of the target tissue experienced from application of the multiple rectangular pulse waves to generate a voltage profile; and
determining the parameter values of the linear model based on the voltage profile and the linear model.

15. The method of claim 6, wherein determining the forward model of the target tissue based on the linear model includes determining the forward model in a frequency domain by dividing the voltage across the target tissue by a total transfer function of the linear model.

16. The method of claim 6, wherein determining the inverse model of the target tissue based on the forward model includes determining a mathematical inverse of the forward model in a frequency domain.

17. The method of claim 1, wherein the filtered stimulation waveform reduces voltage distortion caused by tissue permittivity, tissue capacitance, or both as compared to the stimulation waveform.

18. A method of determining an inverse filter for use in stimulation therapy of tissue, the method comprising:
determining a linear model to approximate an electrode of a therapy device to apply a stimulation waveform to target tissue to be treated and to approximate the target tissue;
determining parameters values of the linear model based on measured voltages during application of a measurement waveform;
determining a forward model of the target tissue based on the parameter values; and
determining an inverse model of the target tissue based on the forward model of the target tissue.

19. The method of claim 18, further comprising:
filtering a simulation waveform based on the inverse model; or
transmitting inverse model information to another device, the inverse model information indicating the inverse model of the target tissue.
